(19) United States
(12) Patent Application Publication
(10) Pub. No.: US 2024/0225040 A9
(43) Pub. Date: Jul. 11, 2024

(54) PULSE PROTEIN GELATION

(71) Applicant: THE GOVERNORS OF THE UNIVERSITY OF ALBERTA, Edmonton (CA)

(72) Inventors: Lingyun CHEN, Edmonton (CA); Roopesh Mohandas Syamaladevi, Edmonton (CA)

(21) Appl. No.: 18/546,637

(22) PCT Filed: Feb. 16, 2022
(86) PCT No.: PCT/CA2022/050223

§ 371 Date: Aug. 16, 2023

Related U.S. Application Data
(60) Provisional application No. 63/149,732, filed on Feb. 16, 2021.

(51) Int. Cl. A23J 3/14; A23J 3/22; A23L 15/00; C07K 1/12

(52) U.S. Cl. CPC A23J 3/14; A23J 3/227; A23L 15/35; C07K 1/12

(57) ABSTRACT

Disclosed are methods of treating a pulse protein to produce a pulse protein gel include the steps of treating the pulse protein (i) with atmospheric cold plasma (ACP); or (ii) by shifting to an alkaline pH or an acidic pH, followed by adjusting the pH back to neutral; or (iii) with both ACP and pH shifting, as well as gels and food additives formed from treated pulse proteins.

### FIELD OF THE INVENTION

The present invention relates to gels formed from pulse protein, and methods of making and using such gels.

### BACKGROUND

One important functional property of food proteins are their gelling capacity to provide food texture. Generally, thermal gelation of globular proteins involves unfolding of the protein by heating, which leads to exposure of active side chains Later, unfolded molecules re-arrange and aggregate via disulfide bridges, hydrogen bonds, hydrophobic and/or van der Waals interactions. Finally, aggregates associate to form a three-dimensional gel network (Clark, Kavanagh, & Ross-Murphy, 2001). Plant proteins usually have weaker gelling capacity compared to animal proteins (e.g., collagen, gelatin, and egg protein) because of their lower solubility and higher denaturation temperature (Liang & Tang, 2013).

Pea protein as an alternative for soy protein has attracted growing interest in food industries, due to its high nutritional value and hypoallergenic status. However, high temperature (>95° C.) is required to enable heat-induced gelation and its gelling properties are relatively weak. Currently, pea protein concentrates and isolates are used as food supplements for athlete diets and functional ingredients in food formulations due to their functional properties (Moreno, Dominguez-Timón, Diaz, Pedrosa, Borderias, & Tovar, 2020).

When compared to plant-based protein like soybean protein, which is more commonly used in food formulations, pea legumin is found to show a relatively weaker gelation performance compared to soybean glycinin due to a lower cysteine content, which contributes to the formation of disulfide bonds and a higher minimum gelling concentration was required for pea legumin (O'Kane, Happe, Vereijken, Gruppen, & van Boekel, 2004).

There is a need in the art for methods of forming pea protein gels which may enable wider applications of pulse protein applications in food formulations where gelling and texturization functions are required. It may be advantageous to enable a pulse protein such as pea protein to be used as a gelling ingredient of plant protein-based food products, such as meat analogues and egg alternatives.

This background information is provided merely to provide information believed to be relevant to a basic understanding of the present invention. It is not an admission that any of the foregoing is prior art against any aspect of the claimed invention.

### SUMMARY OF THE INVENTION

Aspects of the present invention relate to pulse protein gels, and methods of forming such gels. In some embodiments, the pulse protein gels are formed using either cold plasma as a novel non-thermal technique to improve the gelling properties of pea protein, or by pretreatment with a pH-shifting method, or both cold plasma and pH-shifting.

In one aspect, the invention may comprise a method of treating a pulse protein to produce a pulse protein gel, comprising the steps of:
(a) treating the pulse protein
   (i) with atmospheric cold plasma (ACP); or
   (ii) by shifting to an alkaline pH greater than pH 9.0 or an acidic pH less than about pH 5.0, followed by adjusting the pH back to neutral; or
   (iii) with both ACP and pH shifting.

In some embodiments, the pulse protein comprises protein extracted from peas (Pisum sativum), chickpeas, lentils, or beans.

In some embodiments, the invention may comprise one or more of the following features. The pulse protein may be treated by shifting to the alkaline pH, optionally with ACP treatment, either sequentially in either order or simultaneously. The alkaline pH may be between about pH 10.0 to about 12.0. The protein may be held at the alkaline pH for longer than about 1 hour. The protein may be held at the alkaline pH for about 48 hours. The length of ACP treatment may be greater than about 2 minutes and less than about 20 minutes. The protein may be treated by both ACP and pH shift to about pH 12.0. The protein may be treated by both ACP and pH shift with a total treatment time of between about 2 to about 20 minutes.

In some embodiments, the method may comprise the further step of heating the protein to induce gelation, preferably below about 95° C., about 85° C. or about 80° C.

In some embodiments, the method results in a gel having a polymer-like microstructure, having a minimum gel compressive strength of 1.0 kPa, preferably 2.0 kPa, or more preferably 3.0 kPa, which preferably may be achieved without incorporation of an additional cross-linking agent or non-pulse protein.

In another aspect, the invention may comprise a gel formed from pulse protein, said gel having a filamentous, polymer-like microstructure, having been formed from pulse protein treated with atmospheric cold plasma and/or a pH shift to an alkaline pH greater than pH 9.0, preferably about pH 10 to about 12, or an acidic pH lower than pH 5.0, preferably about pH 1.5 to about 3.5, followed by adjusting the pH back to neutral. In some embodiments, the gel has a minimum gel compressive strength of 1.0 kPa, preferably 2.0 kPa, or more preferably 3.0 kPa, after being formed at a gelation temperature below about 95° C., about 85° C. or about 80° C.

In another aspect, the invention may comprise a food additive comprising pulse protein which has been treated with atmospheric cold plasma and/or a pH shift to an alkaline pH greater than pH 9.0, preferably about pH 10 to about 12, or an acidic pH lower than pH 5.0, preferably about pH 1.5 to about 3.5, followed by adjusting the pH back to neutral. In some embodiments, the additive may comprise a powder or a liquid suspension.

### BRIEF DESCRIPTION OF THE DRAWINGS

FIG. 1. Mechanical properties and water holding capacity of pea protein concentrate (PPC) gels made from PPC suspension treated by atmospheric cold plasma (ACP) (12 wt. %; Treated conditions: 3500 Hz, 10 μs, 10 min, 0-30 kV, and 0-1 A) heated at different temperatures (70, 80, and 90° C.) for 30 min. (A) Typical compressive stress-strain curves and photographs of corresponding gels (Left: Untreated PPC gels; Right: ACP treated PPC gels). (B) Compressive strength and Young's modulus values of gels. (C) Frequency dependence of storage modulus (G′) and loss modulus (G″) of PPC suspensions and gels. (D) Water holding capacity of PPC gels. Different letters represent significant differences (p<0.05).

FIG. 2. Transmission electron microscopy (TEM) images of pea protein concentrate (PPC) suspensions with (B) or without (A) atmospheric cold plasma (ACP) treatment. Scanning electron microscopy (SEM) images of PPC gels prepared from PPC suspension treated by ACP at different heating temperatures for 30 min. (C) 70° C., (D) 80° C. and (E) 90° C. . ACP treated conditions: 3500 Hz, 10 μs, 10 min, 0-30 kV, and 0-1 A.

FIG. 3. Effect of simulated gelation process on storage modulus (G′) and loss modulus (G″) of pea protein concentrate (PPC) suspensions (12 wt. %) with or without atmospheric cold plasma (ACP) treatment (3500 Hz, 10 μs, 10 min, 0-30 kV, and 0-1 A) during heating from 25 to 90° C. at a rate of 2° C./min, holding 30 min at 90° C., then cooling from 90 to 4° C. at a rate of 4° C./min and holding 30 min at 4° C.

FIG. 4. Gel dissociation results. (A) Photos of pea protein concentrate (PPC) gels after immersing in different dissociation reagents for 48 h. (B) Frequency dependence of storage modulus (G′) and loss modulus (G″) of PPC gels after immersing in different dissociation reagents for 48 h.

FIG. 5. Effect of atmospheric cold plasma (ACP) treatment (3500 Hz, 10 μs, 10 min, 0-30 kV, and 0-1 A) on the tertiary structure of pea protein concentrate (PPC). (A) Fluorescence intensity of PPC suspensions with or without ACP treatment. (B) Surface hydrophobicity (H0) of PPC suspensions with or without ACP treatment. Different lower-case letters within a column denote significant differences (p<0.05).

FIG. 6. Deconvoluted Fourier transform infrared (FTIR) spectra of the amide I band (1600-1700 cm−1) of untreated and ACP-treated pea protein concentrate (PPC) suspensions (12 wt. %) and gels using ACP-treated PPC suspension (Treated conditions: 3500 Hz, 10 μs, 10 min, 0-30 kV, and 0-1 A) formed at 70, 80, and 90° C. for 30 min, respectively.

FIG. 7. Schematic illustration of the gelling mechanism of atmospheric cold plasma (ACP) enabled pea protein gelation at reduced temperatures (70, 80, and 90° C.). The yellow represents the native pea protein, the green represents the aggregates formed after ACP treatment, and the grey represents the protein molecules with secondary and/or tertiary structure unfolded.

FIG. 8. Mechanical properties of pea protein gels prepared with different treatments: A) 17% HO1-HE2; B) 17% HO24-HE1; C) 17% HO24-HE2; D) 17% HO48-HE2; E) 21% HO48-HE2 (different letters on columns indicate significant difference at p<0.05).

FIG. 9. Rheological properties of pea protein gels prepared with different treatments: Storage modulus (G′) and loss modulus (G″) of pea protein gels as functions of frequency.

FIG. 10. SEM images of 3D pea protein coagulum at pH 12 and gels prepared during and after different treatments: 3D coagulum after a) 1 h holding, c) 24 h holding, e) 48 h holding; gels from b) 17% HO1-HE2, d) 17% HO24-HE2, f) 17% HO48-HE2.

FIG. 11. Water holding capacity (WHC) and swelling ratio of pea protein gels prepared with different treatments: A) 17% HO1-HE2; B) 17% HO24-HE1; C) 17% HO24-HE2; D) 17% HO48-HE2; E) 21% HO48-HE2 (different letters on columns indicate significant difference at p<0.05).

FIG. 12. Viscoelastic response of pH-shifted pea protein slurry (17 wt % protein concentration) at pH 7 with different holding time treatments: (a) 1 h holding, (b) 24 h holding, and (c) 48 h holding during heating and cooling cycle. All the tests were performed at neutral pH.

FIG. 13. FTIR spectra of 3D pea protein coagulum at pH 12 and gels at pH 7 prepared during and after different treatments: a) 14 wt % PPI solution; 3D coagulum after b) 1 h holding, c) 24 h holding, d) 48 h holding; e) 17% HO24-HE2 gel (freeze-dried).

FIG. 14. Frequency dependence of storage modulus (G′) of pea protein gels (17 wt % protein concentration) formed at different holding and heating time before and after soaking in various dissociation reagents for 48 h.

FIG. 15. Scheme of a gel formation mechanism for pea protein treated with pH shifting.

FIG. 16. Effect of pea protein concentrate (PPC) suspensions (PPC_{untreated}) with pH-shifting alone (PPC_{pH-shifting}), atmospheric cold plasma (ACP) treatment alone (PPC_{ACP}) or combined ACP and pH-shifting treatment (PPC_{treated}) on the protein secondary structure (A) tertiary structure (B), molecular weight (C) (M is marker; Lane 1 and 5 are PPC_{untreated} under non-reducing and reducing conditions, respectively; Lane 2 and 6 are PPC_{pH-shifting} under non-reducing and reducing conditions, respectively; Lane 3 and 7 are PPC_{ACP} under non-reducing and reducing conditions, respectively; Lane 4 and 8 are PPC_{treated} under non-reducing and reducing conditions, respectively; LOX, lipoxygenase; CV, convicilin; Lαβ, legumin αβ; V, vicilin; Lα, legumin α; Lβ, legumin β.), surface hydrophobicity (D), and morphology of the protein suspensions (E). Different lower-case letters denote significant differences (p<0.05).

FIG. 17. Effect of pea protein concentrate (PPC) suspensions (PPC_{untreated}) with pH-shifting alone (PPC_{pH-shifting}), atmospheric cold plasma (ACP) treatment alone (PPC_{ACP}) or combined ACP and pH-shifting treatment (PPC_{treated}) on the protein solubility (A) viscosity (B). Different lower-case letters denote significant differences (p<0.05).

FIG. 18. Photographs of gels made from 14 wt. % or 18 wt. % of pea protein concentrate (PPC) suspension (PPC_{untreated}) treated by pH-shifting alone (PPC_{pH-shifting}) or atmospheric cold plasma (ACP) treatment at alkaline pH (PPC_{treated}) heated at different temperatures (70, 80, 90, and 95° C.) for different times (10, 20, and 60 min).

FIG. 19. Mechanical properties and water holding capacity of pea protein concentrate (PPC) gels made from PPC suspension with (PPC_{treated}) or without (PPC_{untreated}) atmospheric cold plasma (ACP) treatment in combination with pH-shifting heated at different temperatures (70, 80, 90, and 95° C.) for different times (10, 20 and 60 min). (A) Typical compressive stress-strain curves. (B) Compressive strength and compressive strain of gels at broken. (C) Frequency dependence of storage modulus (G′) and loss modulus (G″) of PPC suspensions and gels. (D) Water holding capacity of gels. Different letters represent significant differences (p<0.05).

FIG. 20 Scanning electron microscopic (SEM) images of pea protein concentrate (PPC) gels made from PPC suspension with (PPC_{treated}) or without (PPC_{untreated}) atmospheric cold plasma (ACP) treatment in combination with pH-shifting heated at different temperatures (70, 80, 90, and 95° C.) for different times (10, 20 and 60 min). The prepared gel samples were labelled according to their preparation conditions, for example, T-70-10 and N-95-60 represent the gels prepared from PPC_{treated} at 70° C. for 10 min, and PPC_{untreated} at 95° C. for 60 min, respectively.

FIG. 21. Effect of simulated gelation process on storage modulus (G′) and loss modulus (G″) of pea protein concentrate (PPC) suspensions (14 wt. %) with (PPC_{treated}) or without (PPC_{untreated}) atmospheric cold plasma (ACP) treatment in combination with pH-shifting during heating from 25 to 70 (A), 80 (B) and 90° C. (C) at a rate of 2° C./min, holding 10 min at 70, 80 and 90° C., then cooling to 4° C. at a rate of 4° C./min and holding 20 min at 4° C. The temperature is depicted in grey.

FIG. 22. (A-D) Gel dissociation results and (E) deconvoluted Fourier-transform infrared (FTIR) spectra of the amide I band (1600-1700 cm⁻1) of PPC gels. Photos of different pea protein concentrate (PPC) gels after immersing in different dissociation reagents for 48 h (A1, B1, C1 and D1). Frequency dependence of storage modulus (G′) and loss modulus (G″) of original PPC gels and gels after immersing in water and 0.6 M of 2-mercaptoethanol (2-ME) reagents without 6 M urea and 3% sodium dodecyl sulfate (SDS) reagents (due to degradation of gels) for 48 h (A2, B2, C2 and D2).

FIG. 23. The proposed gelation mechanism with low temperature and short time of pea protein treated by atmospheric cold plasma (ACP) in combination with pH-shifting method. Size and distance are not to scale.

### DETAILED DESCRIPTION

Aspects of the present invention relate to methods of forming pulse protein gels using a non-thermal plasma, referred to herein as atmospheric cold plasma (ACP), as a novel non-thermal technique to improve the gelling properties of pulse protein, or by pretreatment with a pH-shifting method, or both ACP and pH shifting. The gels may be formed without the use of other cross-linking agents or other non-pulse proteins.

The pulse protein gels described herein may allow wider applications of pulse protein as an alternative to animal or soy protein in food additives, such as to provide gel-like food texture for meat analogues and egg replacers. Furthermore, treated pulse protein powder may be conveniently incorporated into many food formulations.

In some embodiments, the pulse protein may comprise protein extracted from peas such as yellow or green peas (Pisum sativum); chickpeas; lentils such as red or green lentils; beans such as black beans, red kidney beans, Adzuki beans, black gram beans, navy beans, or broad beans (Faba beans); or any other pulses. Those skilled in the art would recognize a pulse as being defined or recognized by the United Nations Food and Agriculture Organization.

As used herein, a “gel” is a semi-solid comprising a substantially dilute cross-linked system, which exhibits no flow when in the steady-state, although the liquid phase may still diffuse through this system. Typically, globular proteins form 2 types of gels, particulate gels and filamentous gels. The preferred gels described herein are different from typical protein gel microstructures. They exhibit a “polymer-like microstructure” which means the gel network is highly cross-linked, in like manner to polysaccharides or synthetic polymers.

While native pea protein concentrate (PPC) (12 wt. %) does not form a gel under 90° C., ACP-treated PPC show satisfactory gelling properties when heated at a temperature between about 70° and 99° C. The gels exhibit homogeneous three-dimensional network structure with interconnected macropores, and those prepared at higher temperatures, eg. 80 and 90° C., possess good mechanical strength and viscoelasticity, as well as high water holding capacity. ACP treatment contributed to the formation of protein fibrillar aggregates, and significantly reduced the PPC denaturation temperature, leading to protein unfolding at reduced temperature of about 80-90° C. ACP treatment increases the protein surface hydrophobicity and exposes free sulfhydryl groups but decreases total free sulfhydryl groups indicating increased disulfide bonds, which allowed the formation of gels with improved mechanical properties.

Heat-induced gels from pulse protein may also be formed by pre-treatment with a pH-shifting method. Exposing the protein to an acidic or alkaline pH causes the formation of a three-dimensional (3D) coagulum, and the coagulum maintains its structure when pH is adjusted back to neutral (less than about 8, or greater than about 6). Heating the pH-shifted protein above about 70° C., for example at 92° C., led to gels of dense cross-linked polymer-like network with thin connective walls and a small pore size, e.g. 1-2 μm. Such uniform polymer-like gel network microstructure is believed to be related to a high level of protein unfolding during pH-shifting treatment, which made the protein chain flexible and created more active sites to promote molecular interactions including hydrogen bonds and hydrophobic interactions when the protein was neutralized and heated.

The pulse protein gels formed by the methods described herein exhibit excellent mechanical properties and water holding content (WHC) comparable to gels made from some animal proteins or soy protein.

The pulse protein may be supplied as a powder. In one example pea protein powder (for example, 53.1% protein, 33.7% carbohydrate, 2.0% fat, 5.3% ash, and 5.9% moisture) was provided by AGT Foods Research & Innovation Centre (Saskatoon, SK, Canada). Pea protein concentrate (PPC) may be prepared by a washing process, substantially as described by Walia and Chen (2020). Briefly, pea protein powder is mixed with distilled water to form a suspension, preferably at a 1:7 ratio (w/v). The pH of the suspension may be adjusted as desired, for example to pH 9 with 1 M NaOH. After stirring, the solid fraction may be collected, such as by centrifuging or filtration. The solid fraction may then be dispersed in distilled water and adjusted to pH 7 by adding 1 M NaOH. Next, the mixture was dialyzed (3.5 kDa MWCO dialysis tubing) against distilled water for three days and then dried or lyophilized to obtain the PPC powder. The protein content of the powder may preferably be between about 70-95% (w:w).

### Atmospheric Cold Plasma Treatment

Some embodiments of the present invention are based on the use of atmospheric cold plasma (ACP) as a treatment to improve pea protein gelling properties. As used herein, a “plasma” is a fully or partially ionized gas containing neutrals, ions, free radicals and electrons, which can be produced by a variety of electrical discharges.

ACP is well-known to those skilled in the art, and is conventionally used as an alternative to traditional thermal processing for microbial inactivation to extend the shelf life of different food products such as fruits, vegetables, meats, eggs, and cereals (Miao, Nyaisaba, Koddy, Chen, Hatab, & Deng, 2020). ACP technology has the advantages of shorter treatment time and no thermal damage to food physical properties (e.g., color, texture), flavor and nutritive value (e.g., vitamins and flavonoids) due to its lower temperature (<60° C.) compared to traditional thermal processing (Deng, Ruan, Mok, Huang, Lin, & Chen, 2007; Mandal, Singh, & Singh, 2018).

At atmospheric pressure, when air is used, an ACP process generates reactive oxygen species (ROS) such as O₃, O, O⁻, O₂⁻, O₂⁻², H₂O₂ or —OH, and reactive nitrogen species (RNS) such as NO, NO₂ or N₂. These reactive species are known to trigger lipid peroxidation in microbial cell membranes, and oxidize proteins and DNA, leading to microbial inactivation (Feizollahi, Misra, & Roopesh, 2020). The active species generated by ACP can also break covalent bonds and initiate some chemical reactions (Kim, Lee, & Min, 2014). ACP treatment can expose active sites on the protein surface, thus increasing the affinity between protein and water molecules. The enhanced surface polarity of the protein molecules by ACP treatment is associated with the improved solubility (Dong, Gao, Xu, & Chen, 2017; Ji, et al., 2018; Ji, et al., 2019). In addition, ACP technology could affect the formation of disulfide bonds in proteins.

ACP may be applied with suitable plasma systems, which are generally commercially available. For example, a dielectric barrier discharge plasma system (PG 100-D, Advanced Plasma Solutions, Malvern, USA) is representative of an exemplary suitable system.

In some embodiments, a pea protein suspension (such as 12 wt. %) may be prepared by dispersing and stirring protein powder in water, preferably ensuring thorough hydration. The protein suspension is then placed between a high-voltage electrode and a ground electrode of a plasma system. The distance from the sample surface to the high-voltage electrode may between about 1 mm to about 5 mm, preferably between about 2 mm and 4 mm. The frequency may be between about 1000 Hz and 5000 Hz, preferably between about 2000 to about 3500 Hz, such as about 3000 Hz. The duty cycle may be varied, such as 70%. The voltage output may be 2-10 kV, 0-20 kV, or 0-30 kV, the current output may be 0.0 to 1.0 A. The pulse width may be 10 μs. The length of treatment may vary between seconds and minutes, for example between about 2 to about 20 min, preferably between about 5 to about 15 min, such as 10 min. The process may be repeated, for example five times. The temperature of ACP-treated sample may slightly increase as a result of such treatment, for example 1-3° C., however, the process is preferably non-thermal in that the suspension is not heated.

The ACP-treated PPC may optionally be processed to remove air bubbles, and heated at gelation temperature between about 70° and about 100° C., preferably above 70° C., followed by cooling, to strengthen the gel network. At 70° C., the protein does not significantly unfold and the undenatured protein may form particulate aggregates to build up a coarse and particulate network together with the initially formed fibrillar aggregates by ACP treatment, leading to self-standing but relatively weak gels (the compressive strength of 0.53 kPa). At about 80° C. and above, ACP treated pea protein unfolds to develop a strong fibrillar network mainly stabilized by hydrogen bonding together with other interactions. This resulted in a gel with a compressive strength of 2.70 kPa, a water holding capacity of 85%, and good viscoelasticity. At 90° C., a stronger gel with a more homogenous porous structure was formed because the more fully unfolded protein exposed more hydrophobic groups and active sites. In this way, protein chains were crosslinked to the fibrillar aggregates formed by ACP treatment through more interactions. In preferred embodiments, the gel strength reaches 6.27 kPa, water holding capacity is 88%, and with good viscoelasticity.

Without restriction to a theory, it is believed that the gelling mechanism of atmospheric cold plasma (ACP) treated pulse protein is based on the protein tertiary structure partially unfolding, exposing internal hydrophobic side chains and active sites. The active groups on the surface of protein molecules (such as —SH groups) may react with some free radicals generated by ACP to form covalent bonds (e.g., —S—S— bonds). At the same time, the ACP treated pea protein concentrate (PPC) suspension contained a significant amount of negative charge, which prevents protein random aggregation, thus the protein grows linearly based on hydrophobic patches, to form fibrillar aggregates.

### pH-Shifting

Protein conformational changes are sensitive to pH variations due to electrostatic repulsion. As used herein, “pH-shift” or “pH-shifting” means to treat the protein with acidic or alkaline conditions sufficient to substantially disrupt the tertiary structure of the protein. Thus, in some embodiments, pulse proteins may be treated by exposure to alkaline pH (greater than pH 9.0, preferably about pH 10 to about 12) or acidic pH (lower than pH 5, preferably about pH 1.5 to about 3.5), followed by adjusting the pH back to neutral. Heating the pH-shifted proteins induces gel formation. The shift to an alkaline or acidic pH induces an intermediate conformational state during protein unfolding, which has a similar secondary structural content as the native state, but with a disrupted tertiary structure.

When pulse protein is exposed to more alkaline conditions, for example pH 12, more extensive protein unfolding and disruption of disulfide bonds occurred to expose hydrophobic side chains. Pulse protein also loses some side-chain interactions to create free active sites, thus becoming more flexible. The increase of ionic interactions and hydrogen bonds between charged proteins and water contributes to improved protein solubility.

The pH-shifting process preferably comprises treatment at a highly alkaline pH, e.g. greater than pH 10, such as pH 12, because the higher pH significantly improved pea protein solubility and emulsifying properties when compared to other samples modified by pH-shifting at pH 2, 4, and 10. It is believed the protein underwent more unfolding under pH 12 for modification of functional properties.

Without restriction to a theory, it is believed that pulse protein molecules which exist as mostly hexamers and trimers, dissociate to individual subunits upon alkali or acid treatment. The subunits partially unfold to facilitate protein aggregation to form initial protein crosslinking. When pH is adjusted back to neutral, certain refolding is expected due to neutralization of the negative charges. The refolding of the protein upon neutralization was negatively correlated to the degree of unfolding when exposed to alkaline pH, so the extent of unfolding in protein coagulum treated for an extended period (48 h holding) was expected to be the highest. Polymer-like gels develop upon heating because partial protein unfolding, and crosslinked aggregates were developed prior to heating. Further unfolding and protein aggregations both took place during heating, where extensively unfolded protein developed at a lower heating temperature, which might not quickly induce aggregations so that polymer-like gels with uniform network structure develop. Heating native protein molecules without pH-shifting pre-treatment results in unfolding at a higher temperature followed by quick aggregation, thus are more likely to form gels with particulate microstructures.

The uniform polymer-like network structure which results contributes to strong and elastic gels when compared to particulate gels obtained from native untreated protein directly. In addition, such a polymer-like network created more active sites to promote protein molecular interactions, resulting in gels enforced by improved hydrogen bonds and hydrophobic interactions to withstand large deformations. Moreover, the fine-stranded polymer-like gels exhibited evenly distributed network with small pore size, giving high water holding capacity and minimum water loss.

### Combined ACP and pH Shifting

In some embodiments, the invention may comprise a method of forming a pulse protein gel, comprising the step of treating pulse protein with ACP and pH-shifting, simultaneously or in either order. Satisfactory pulse protein gels may be formed at reduced temperatures (e.g., as low as 70° C.) that are lower than the denaturation temperature, even with relatively short heating times (e.g., 10-20 min), and shorter exposure to the combined ACP and pH-shifting treatment (e.g. 10 min) than with either treatment individually.

The same factors and features described herein for each treatment individually applies when the treatments are combined. In some embodiments, the combined treatment may permit significantly shorter treatment time when the treatments are applied, either consecutively or simultaneously. The resultant gels from the combined treatment show strength comparable to soy protein gels.

### EXAMPLES

Specific embodiments of the present invention are described with reference to the following Examples. These Examples are provided for the purpose of illustration only.

### Examples Section 1—ACP Treatment

### Materials and Chemicals

Pea protein powder (53.1% protein, 33.7% carbohydrate, 2.0% fat, 5.3% ash, and 5.9% moisture) was provided by AGT Foods Research & Innovation Centre (Saskatoon, SK, Canada). Sodium dodecyl sulfate (SDS), 2-mercaptoethanol (2-ME), 5,5′-dithiobis-(2-nitrobenzoic acid) (DTNB), 1-anilinonaphthalene-8-sulfonic acid (ANS), 2,4,6-trinitrobenzene sulfonic acid (TNBS) (5% (w/v) in H2O), glycine, L-lysine, Trizma® base and ethylenediaminetetraacetic acid (EDTA) were purchased from Sigma-Aldrich (St. Louis, MO, USA), urea and other chemicals (analytical grade) were purchased from Fisher Scientific (Fair Lawn, NJ, USA).

Preparation of Pea protein Concentrate (PPC)

PPC was prepared as described by Walia and Chen (2020) with minor modifications. Briefly, pea protein powder was mixed with distilled water at a 1:7 ratio (w/v) and the pH of the suspension was adjusted to 9 using 1 M NaOH. After stirring for 1 h at room temperature, the mixture was centrifuged at 8000×g for 15 min (Acanti® J-E centrifuge, Beckman Coulter, USA). The supernatant was collected and adjusted to pH 4.5 using 1 M HCl. Then the suspension was centrifuged at 8000×g for 15 min and the precipitate was collected. The pellet was dispersed in distilled water and adjusted to pH 7 by adding 1 M NaOH. Next, the mixture was dialyzed (3.5 kDa MWCO dialysis tubing) against distilled water for three days and then lyophilized to obtain the PPC powder. The protein content was 87.2% measured by a Leco nitrogen analyzer (Leco®, USA) using a nitrogen-to-protein conversion factor of 5.96 (Fujihara, Kasuga, & Aoyagi, 2001).

### Atmospheric Cold Plasma (ACP) Treatment

ACP with a dielectric barrier discharge plasma system (PG 100-D™, Advanced plasma solutions, Malvern, USA) was used to treat the PPC sample. Pea protein suspension (12 wt. %) was prepared by dispersing PPC (1.2 g) in distilled water (8.8 g) with magnetic stirring (700 rpm) at room temperature (22-23° C.) for 2 h and then stirred overnight at 4° C. in refrigerator to ensure thorough hydration. In the next day, the PPC suspension was taken out from the fridge (4° C.) to return to room temperature before further experiments. Approximate 2 mL of 12 wt. % PPC suspension in a 4-cm diameter and 0.5-cm height plastic container (METER®, Pullman, WA, USA) was placed between a high-voltage electrode and a ground electrode. The distance from the sample surface to the high-voltage electrode was maintained at 2 mm, the frequency was 3500 Hz and the duty cycle was 70%. The voltage output was 0-30 kV, the current output was 0-1 A, and the pulse width was 10 μs. The sample was treated for 2 min, followed by well stirring. This process was repeated five times, so the total treatment time was 10 min. The temperature of ACP-treated sample increased from 21.3° C. to 23.5° C.

### Preparation of PPC Gels and Strength

Approximately 5 mL of ACP-treated PPC (12 wt. %, pH 6.0) was transferred into a 10 mL (BPA-free) of cryovial (Simport™ polypropylene T310-10A) at room temperature. All tubes were centrifuged at 3000 ×g for 1 min (Centrifuge 5810 R, Eppendorf, Germany) to remove air bubbles, and then heated at 70, 80, and 90° C. for 30 min followed by cooling in an ice-water bath for 10 min, and then stored in a fridge (4° C.) for 24 h prior to analysis. The gels were also prepared from untreated PPC suspension (pH 6.9) at the same concentration and temperature for comparison. Each sample was replicated three times.

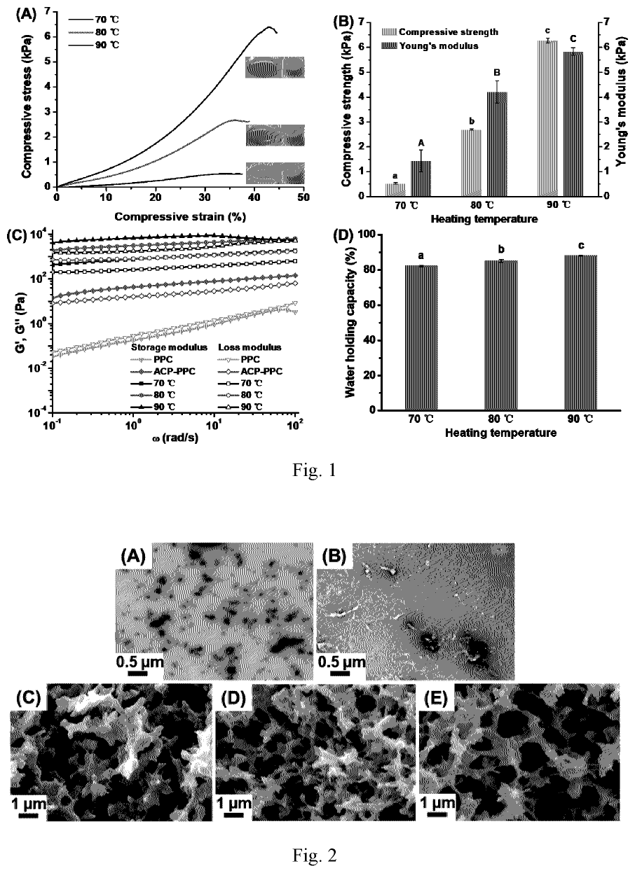

The impact of various ACP treatments on pea protein properties, and suitable ranges and optimized conditions have been determined (3500 Hz, 10 μs, 10 min, 0-30 kV, and 0-1 A) which can improve the gelling capacity of pea protein. As shown in FIG. 1A native PPC suspension (12 wt. %) could not form a gel after heating at 100, 110, or even 120° C. for 30 min. Even though the protein concentration was increased to 15 wt. %, a very weak gel was formed after heating at 95° C. for 30 min, and no gels were formed at temperature below 95° C.

After ACP treatment, PPC was found to form gels even at 70° C., which was significantly lower than the denaturation temperature (legumin of around 92° C.) of pea protein (Liang, et al., 2013). FIG. 1A and B show the impact of heating on the mechanical properties of the gels formed from ACP-treated PPC.

Mechanical properties of gel samples were measured using an Instron 5967 universal testing instrument (Instron Corp., MA, USA) equipped with a 50 N load cell (Yang, Wang, Vasanthan, & Chen, 2014). The gel samples had a height of 8 mm and a diameter of 14 mm. The sample was compressed to 30% of its original height with a rate of 1 mm/min at room temperature (22-23° C.). Compressive strength was calculated as the maximum compressive stress in the stress-strain curve. Young's modulus was determined by the slope of the linear section (the compressive strain of 0-10%) in the stress-strain plots.

The gel formed at 70° C. showed a compressive strength of 0.53 kPa, and this value increased to 2.70 kPa and 6.27 kPa when the temperature was raised to 80° C. and 90° C., respectively. The Young's modulus of the gel formed at 90° C. was 5.84 kPa, followed by gel heated at 80° C. (4.21 kPa) and 70° C. (1.43 kPa). It was noted that the gels formed at 80° C. from 12 wt. % ACP treated PPC showed even higher mechanical strength than those of native pea protein gels (15 wt. %) heated at 95° C. for 30 min (1.03 kPa) (Munialo, van der Linden, & de Jongh, 2014).

Young's modulus of the gel formed by ACP-treated PPC (4.21 kPa) was higher than that of gel formed from soy protein isolate at the same conditions (80° C. for 30 min) was higher than that of the gel formed by soy protein isolate (15 wt. %, pH 7, 10 mM CaCl₂) at 80° C. for 30 min (1.84 kPa) (Brito-Oliveira, Bispo, Moraes, Campanella, & Pinho, 2018).

### Viscoelastic Properties

The storage modulus (G′) and loss modulus (G″) indicate the elastic and viscous properties of gels. As shown in FIG. 1C, G″>G′ was observed for native PPC in suspension, indicating liquid behavior dominated in the sample. G′>G″ was observed for ACP-treated PPC suspension along with an almost constant G′ at all frequencies tested, implying a weak gel behavior for pea protein after ACP treatment (Ben-Harb, et al., 2018). But G″-value of ACP treated PPC suspension shows a high dependence on frequency from 10⁻¹ to 10² rad/s, suggesting formation of the entanglement networks which were formed by the simple topological interaction of polymer chains rather than by cross-linking (Picout & Ross-Murphy, 2003). Thus, PPC suspension treated with ACP did not form the solids “true” gels, but structured fluids. The G′ was higher than G″ of the gel samples, indicating that all samples exhibited solid-like behaviors. Both moduli increased with increasing the heating temperature, which indicated that the formed gel became stronger as the heating temperature increased. Higher heating temperature led to an increased degree of protein unfolding, thus more active groups were exposed to facilitate covalent and non-covalent interactions, which brought out stronger three-dimensional gel networks. In addition, the G′ of the gel heated at 90° C. decreased sharply when the frequency exceeded around 10 rad/s as a result of gel break down at large deformation, which is a typical behavior of strong gels (Martinez-Lopez, et al., 2016). This rheological characterization results demonstrate structured fluid formed from PPC by ACP treatment, which was then converted into viscoelastic gels by heating at 70-90° C.

### Morphology of Protein Suspensions and Gels

As ACP-treated PPC gels showed solid-like behavior based on rheological results, the

morphology of PPC with or without ACP treatment was observed by TEM. The PPC in its native state was shown in FIG. 2A, whereas fibrillar structure was observed for ACP treated PPC (FIG. 2B), indicating the formation of fibrillar aggregates of PPC as a result of ACP treatment.

The microstructures of PPC gels prepared at 70, 80, and 90° C. are shown in SEM images (FIG. 2). The 3D gel network was obtained from PPC even at 70° C., but relatively loose structure with uneven pores formed from particulate aggregates was observed (FIG. 2C). When the heating temperature was increased to 80° C., strong 3D gel networks with regular interconnective pores were formed (FIG. 2D). The pores became larger and more homogenous when the gel was formed at 90° C., showing a macro-porous wall network structure (FIG. 2E). The regular interconnective porous network structure explained the good firmness and elasticity of the PPC gels prepared at 80 and 90° C., as well as their high water holding capacity because gels with uniform pore structure possess the good capacity to hold moisture through capillary forces (Wu, Xiong, Chen, Tang, & Zhou, 2009).

### Gelation and Dissociation of Process

Rheological measurements were performed on a TA Discovery HR-3 rheometer (TA Instruments, DE, USA) using a parallel plate geometry with a diameter of 40 mm. Unless described otherwise, all measurements were conducted at a gap of 1 mm by applying a constant strain of 1%. Frequency sweeps were done to determine the storage modulus G′ and loss modulus G″ at oscillation frequency from 0.1 to 100 rad/s. To study the changes in viscoelastic properties as a function of temperature, the PPC suspensions with or without APC treatment were subjected to a temperature ramp at a rate of 2° C./min from 25 to 90° C., held at 90° C. for 30 min, and then cooled down to 25° C., and held at 4° C. for 30 min. Silicone oil was applied to cover the surface of samples to prevent evaporation during the tests. There was no sample weight loss before and after the tests and the effect of silicone oil on the measurements was regarded as negligible. The temperature of the plate was controlled with a Peltier system. All rheological measurements were performed within a predetermined linear viscoelastic region at the strain value at 1%.

Molecular interactions involved in the gel formation were studied by a rheological measurement in presence of dissociation reagents (Nieto-Nieto, Wang, Ozimek, & Chen, 2015; Yang, Wang, Vasanthan, & Chen, 2014). The gel samples were cut into approximately a height of 1.0 cm and a diameter of 1.4 cm. The resulting gel disks were immersed into 0.2 M 2-mercaptoethanol (2-ME), 2 M urea, or 1% (w/v) sodium dodecyl sulfate (SDS) solution for 48 h at room temperature. The gel disks immersed in the distilled water were used as control samples. A frequency sweep analysis was conducted on the soaked gels to measure G′ and G″.

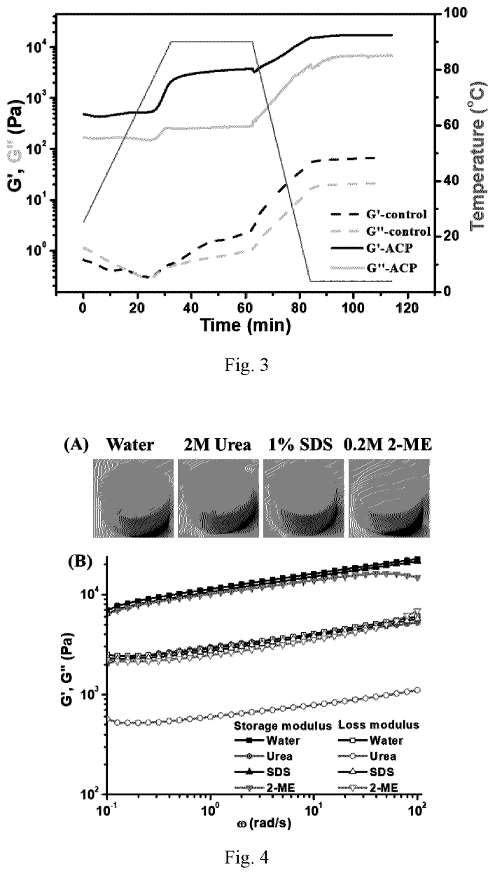

Dynamic rheological properties of the PPC suspensions with or without ACP treatment were studied when subject to a temperature ramp to simulate the gelation process (FIG. 3). G′ and G″ of ACP-treated PPC suspension were much larger than that of untreated PPC suspension. For untreated PPC suspension, G′ was lower than G″ initially, indicating a liquid-like behavior. Both G′ and G″ were reduced from 25° C. to 76° C., likely due to the decrease of hydrogen bonds and electrostatic interactions in protein molecules by initial heating, which resulted in decreased viscosity of the suspensions (Wang, Luo, Zhong, Cai, Jiang, & Zheng, 2017). Untreated PPC started to show solid behavior at 76° C. when G′ and G″ crossed each other, and the G′ value increased from 76 to 90° C. A continuous increase in G′ was observed during holding at 90° C. for 30 min and then in the process of cooling to 25° C. However, in the actual gelation experiment, the native PPC suspension did not form a gel even when heated at 90° C. for 30 min followed by cooling. This suggests that native PPC continued to develop crosslinking during heating and cooling processing, but the protein concentration and heating temperature was not sufficient for it to form a self-standing gel network. For ACP-treated PPC suspension, structured fluid was formed before heating, thus G′ was higher than G″ at the beginning. A decrease in G′ value during heating from 25° C. to 39° C. indicates that the formed protein network structure was interrupted probably due to the breaking of hydrogen bonds at the initial heating step, leading to a loss of firmness. While G′ and G″ values of ACP-treated PPC suspension maintained consistent with increasing temperature from 39° C. to 75° C., G′ increased sharply from 75° C. to 90° C., suggesting gelation started at 75° C. In the second phase when the temperature was maintained at 90° C. for 30 min, G′ of ACP-treated PPC suspension remained almost unchanged, suggesting no further gel-enhancement was occurred during the isothermal stage. Next, when the temperature was decreased from 90° C. to 4° C. in the third phase, G′ increased sharply, indicating that cooling stage further strengthened the 3D gel network. This might be related to increased hydrogen bonds in the gel network, which is favored at lower temperatures.

The gels formed at 90° C. were immersed into dissociation reagents including urea, sodium dodecyl sulfate (SDS) and 2-mercaptoethanol (2-ME) in order to disrupt hydrogen bonds, hydrophobic interactions and disulfide bonds, respectively. As shown in FIG. 4A, there was no apparent change in the appearance of these gels after immersing in water or dissociation reagents for 48 h. FIG. 4B shows the rheological properties of gels soaked in dissociation reagents, and the gels soaked in water were also tested for control. G′ and G″ of gels soaked in urea were significantly lower than the control group, indicating that hydrogen bonds indeed played a major role in the gel network structure.

### Water Holding Capacity (WHC)

Water holding capacity of a gel is an important property for its application in food production because a lack of water has a negative impact on the texture and quality of a foodstuff. FIG. 1D demonstrates good water holding capacity (80-90%) of the gels prepared at all the tested temperatures, which was comparable or even slightly higher than soy protein gels (50-80%) (Braga, Azevedo, Marques, Menossi, & Cunha, 2006; Brito-Oliveira, et al., 2018; Tsumura, Saito, Tsuge, Ashida, Kugimiya, & Inouye, 2005).

Water holding capacity (WHC) was determined according to a method described by Nieto-Nieto et al. (2016) with slight modification. PPC gel samples (1.0 g) were placed into a Vivaspin 20 centrifugal filter unit (GE Healthcare Bio-Sciences AB, Uppsala, Sweden) and centrifuged at 4000×g for 20 min. The weight of the gel was recorded before or after centrifugation. The WHC of gels was calculated according to equation (1):

$$WHC = \frac{{M}_{1} - {M}_{2}}{{M}_{1}} \times 100 \% \qquad ( 1 )$$

Where M₁ represents the mass of total water in the gel before centrifugation (g), M₂ represents the mass of water released in the gel by centrifugation (g).

### Morphology of PPC Gels and PPC Suspensions

The morphology of protein samples before or after ACP treatment in suspension was observed using a transmission electron microscope (TEM, Morgagni 268, Philips-FEI, Hillsboro, USA) at an accelerating voltage of 80 kV. A drop of the diluted PPC suspensions (0.03 wt. %) was transferred onto a carbon film-covered 400 mesh copper grid. Subsequently, a drop of 4% uranyl acetate (staining solution) was added for negative staining for 45 s, then the excess solution was removed with filter paper.

A Zeiss EVO M10 scanning electron microscope (SEM) (Carl Zeiss AG, Oberkochen, Germany) was used to examine the microstructure of the PPC gels. The gel samples were rapidly frozen in liquid nitrogen, and then freeze-dried. The cross-sections of the gel samples were sputter-coated with gold under vacuum for 90 s and then was observed to record the microstructure at 20 kV.

### Surface Charge

The surface charge (zeta potential) of PPC suspensions with or without ACP treatment were determined by laser Doppler velocimetry using a Zetasizer Nano-ZS (Malvern Instruments Ltd., UK). The protein refractive index (RI) was set at 1.45 and dispersion medium RI was 1.33. The samples were properly diluted before measurement.

### Content of Free Sulfhydryl (—SH) and Free Amino (—NH₂) Groups

The contents of free —SH groups including total and exposed —SH were measured using the method described by Dong et al. (2017) with modification. PPC suspensions (12 wt. %) with or without ACP treatment were diluted five times with Tris-glycine buffer (pH 8.0). Then 1.0 mL of the diluted sample suspension was mixed with 2.0 mL of Tris-glycine buffer (pH 8.0) and 0.02 mL of Ellman's reagent (DTNB, 5,5′-dithiobis-(2-nitrobenzoic acid), 4.0 mg/mL) in 5.0 mL of microcentrifuge tubes. After the mixture was incubated at 25° C. for 5 min, the exposed free —SH group content was determined at 412 nm using a SpectraMax M3 microplate reader (Molecular Devices, USA). The total —SH group content was evaluated as well. First, 0.5 mL of diluted samples was mixed with 0.05 mL of 2-mercaptoethanol and 2.5 mL of 10 mol/mL urea in Tris-glycine buffer (pH 8.0) in 15 mL centrifuge tubes. After incubating at 25° C. for 1 h, 2.5 mL of 12% trichloroacetic acid (TCA) was added and the mixture was further incubated for 1 h at 25° C. Subsequently, the tubes were centrifuged at 3000×g for 15 min and the precipitate was suspended in 2.0 mL of 12% TCA. The mixture was centrifuged again to remove the 2-mercaptoethanol. Finally, the precipitate was dissolved in 3.0 mL of 8.0 mol/mL urea in Tris-glycine buffer (pH 8.0) and then 0.05 mL Ellman's reagent was added. After 5 min, the absorbance was read at 412 nm using a SpectraMax M3 microplate reader (Molecular Devices, USA). The content of the free —SH group was calculated according to equation (2).

$$SH ( \frac{µ mol}{g} ) = \frac{73.53 \times A \times D}{C} \qquad ( 2 )$$

Where A is the absorbance at 412 nm; D is the dilution multiple; C is the sample concentration (mg/mL).

The content of free —NH₂ groups was evaluated by the trinitrobenzene sulphonate (TNBS) assay (Habeeb, 1966) with slight modification. The PPC suspensions with or without ACP treatment were diluted with reaction buffer (0.1 M sodium bicarbonate, pH 8.5). And 0.01% (w/v) TNBS solution was prepared using reaction buffer as the diluent. Then, 0.5 mL of the sample was mixed with 0.25 mL of the 0.01% TNBS solution. After incubating at 37° C. for 2 h, 125 μL of 1 N HCl was added to the mixture to terminate the reaction. Then the absorbance of the mixture was recorded at 335 nm using a SpectraMax M3 microplate reader (Molecular Devices, USA). L-lysine solution (2.5, 5, 10, 20, and 40 μg/mL) was used as a standard instead of a sample to repeat the above experimental steps and record absorbance. The content of free —NH₂ groups was calculated by the standard curve.

### Surface Hydrophobicity (H₀)

The surface hydrophobicity (H₀) of samples was evaluated using the fluorescent probe 1-anilinonaphthalene-8-sulfonic acid (ANS) (Kato & Nakai, 1980) according to Chalamaiah et al. (2017) with slight modification. Serial dilutions (protein concentration of 0.001 to 0.5 mg mL⁻¹) of the PPC suspensions with or without ACP treatment were prepared by diluting with 0.1 M phosphate buffer (pH 7.4). Then, 20 μL of 8 mM ANS solution was mixed with 4 mL diluted sample. The fluorescent intensity was measured at an excitation wavelength of 390 nm and an emission wavelength of 460 nm using a SpectraMax M3 microplate reader (Molecular Devices, USA) after the mixture was kept in the darkroom for 15 min. The initial slope of the corrected fluorescence intensity (the fluorescence intensity of protein suspension with ANS subtracted protein suspension without ANS) versus protein concentration plot was calculated by linear regression analysis and used as an index of surface hydrophobicity.

### Chemical Structural Changes

Table 1 summarizes the changes in the surface charge of untreated and ACP-treated PPC suspensions. Since the isoelectric point of pea protein (about pH 4.5) was lower than the pH value of the native PPC suspension (pH 6.9) and the ACP-treated PPC suspension (pH 6.0), both samples were negatively charged as reflected in their zeta potential values. The decrease in pH by ACP treatment might be due to the formation of some acidic substances, such as acidic H₃O⁺ ions formed from the reaction between water molecules and H₂O₂ generated by ACP, and HNO₃ and HNO₂ generated from NO through NO₂ (Ji, et al., 2018). Because the inherent minerals of pea protein caused relatively low salt levels in suspension, the protein molecules aggregated in an ordered ‘string-of-beads’ via the weakened electrostatic repulsion after ACP treatment and possible attractive hydrophobic interactions between protein molecules (Bryant & McClements, 1998).

TABLE 1
Zeta potential of pea protein concentrate (PPC) suspensions
with or without atmospheric cold plasma (ACP) treatment
(3500 Hz, 10 μs, 10 min, 0-30 kV, and 0-1 A).

| Samples | Zeta potential (mV) |
|---|---|
| PPC suspension | −27.3 ± 0.53^{b} |
| ACP-treated PPC suspension | −24.2 ± 0.10^{a} |

Different lower-case letters within a column denote significant differences (p < 0.05), as determined by paired T-test.

On the one hand, protein unfolding can expose free —SH groups that are originally hidden inside the protein globular structure, on the other hand, —SH groups are easily oxidized by reactive oxygen species (ROS) generated by ACP, such as ·OH (Zhang, et al., 2020) and H₂O₂ (Sullivan & Sebranek, 2012). Thus, the changes of the total and exposed —SH groups, as well as the free —NH₂ groups of PPC before and after ACP treatment were measured and the results are displayed in Table 2. ACP treatment reduced the total —SH groups from 10.22 to 8.28 μmol/g protein. However, the content of exposed free —SH groups was slightly increased after ACP treatment. These results suggest protein unfolding to expose more —SH groups, although the generated free —SH groups reacted with reactive species to form —S—S— bonds at the same time. Meanwhile, the free —NH₂ groups of PPC had no significant changes after ACP treating, which means no significant protein hydrolysis occurred.

TABLE 2
The contents of free —SH and —NH₂ groups of pea protein
concentrate (PPC) suspensions with or without atmospheric cold
plasma (ACP) treatment (3500 Hz, 10 μs, 10 min, 0-30 kV, and 0-1 A).

| | | | Free —NH₂ |
|---|---|---|---|
| | Free —SH contents | | contents |
| | (μmol/g protein) | | (μmol —NH₂/ |
| Samples | Total | Exposed | mg protein) |
| PPC suspension | 10.22 ± 0.03^{b} | 3.93 ± 0.03^{a} | 0.59 ± 0.02^{a} |
| ACP-treated PPC | 8.28 ± 0.02^{a} | 4.07 ± 0.04^{b} | 0.61 ± 0.03^{a} |
| suspension | | | |

Different lower-case letters within the same column denote significant differences (p < 0.05), as determined by paired T-test.

### Protein Secondary and Tertiary Structural Change

FTIR spectra of the PPC in suspensions and gels were recorded using a Nicolet 6700 Fourier transform infrared spectrophotometer (Thermo Fisher Scientific Inc., MA, USA). All data were collected in the spectral range from 4000 to 800 cm⁻¹ during 128 scans at a spectral resolution of 4 cm⁻¹. D₂O was used as a solvent for the PPC suspensions and gels preparation. The samples were placed between two CaF₂ windows separated by a 25 μm polyethylene terephthalate film spacer for test. The spectrophotometer was continuously purged with dry air from a lab gas generator (Parker Hannifin Corp., USA). Protein secondary structures (α-helices, β-sheets, β-turns, and random coils) were determined by analyzing the amide I band region (1700-1600 cm⁻¹) according to the Fourier self-deconvolution (FSD) algorithm using Omnic 8.1 software (Thermo Fisher Scientific, MA, USA) at a bandwidth of 24 cm⁻¹ and an enhancement factor of 2.5.

The fluorescence spectra of the PPC suspensions with or without ACP treatment were measured by a SpectraMax M3 microplate reader (Molecular Devices, USA). The samples were properly diluted before measurement. The excitation wavelength was 295 nm. The emission spectra at 300-400 nm were recorded at 10 nm/s scanning speed. The emission and excitation slits were set at 3 nm.

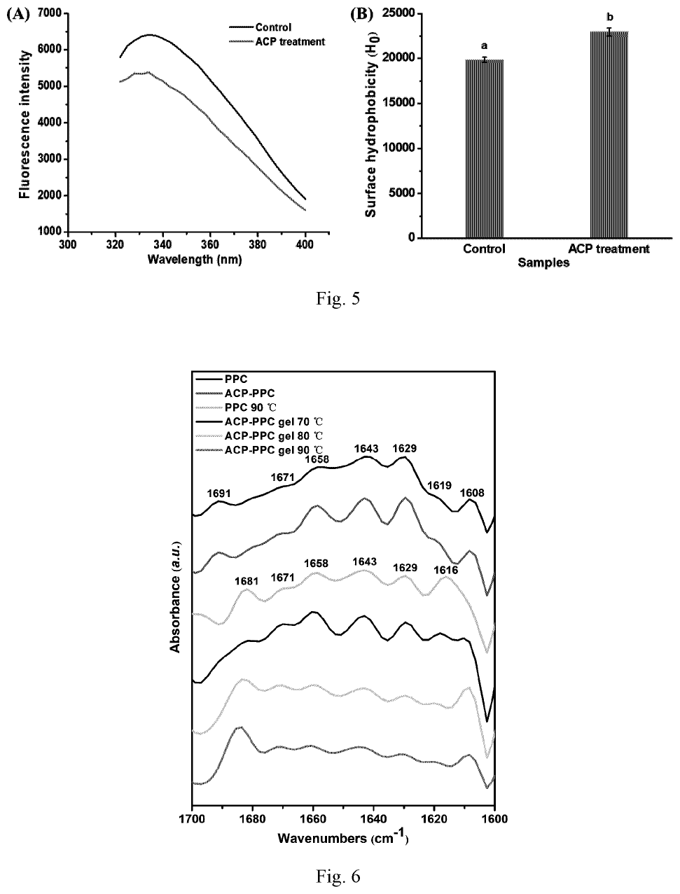

The tertiary structure of proteins can be evaluated by studying intrinsic fluorescence spectra from tryptophan, tyrosine, and phenylalanine residues. FIG. 5A shows the tryptophan fluorescence spectra of PPC with or without ACP treatment. The native PPC showed the maximum fluorescence emission spectrum of the tryptophan residue in the wavelength of near 340 nm. After ACP treatment, the fluorescence intensity decreased significantly, indicating the unfolding of PPC exposed tryptophan to a more hydrophilic environment. It is also possible that tryptophan oxidation occurred during ACP treatment because the hydrogen atom on the tryptophan indole ring could be removed by radicals.

The protein surface hydrophobicity (H₀) was also determined to assess protein denaturation. The H₀ value of PPC suspension increased from 19800 to 23000 after ACP treatment (FIG. 5B), indicating a higher number of ANS binding sites on the PPC surface. But the H₀ did not increase substantially, indicating PPC was only partially unfolded as a result of ACP treatment. Protein oxidation can lead to protein unfolding and denaturation with increased H₀, which might be caused by free radicals that broke the hydrogen bonds or electrostatic interactions in protein molecules (W. Sun, Zhao, Yang, Zhao, & Cui, 2011). Therefore, it is believed that the increase in H₀ of ACP-treated PPC suspension could be caused by oxidation. The exposure of hydrophobic residues was favorable for the formation of the gel network through hydrophobic interactions.

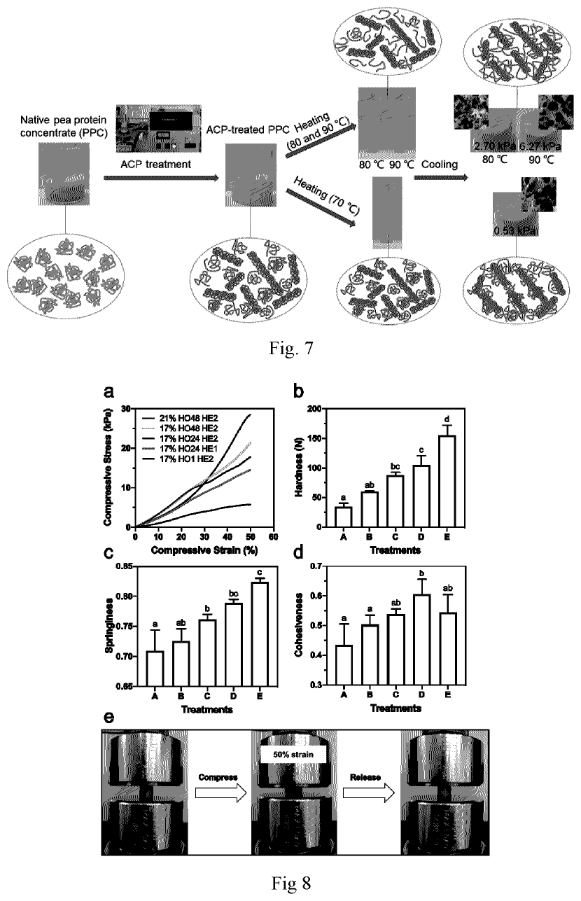

The changes in protein secondary structure were studied by FTIR through the analysis of the amide I band (1600-1700 cm⁻¹) (FIG. 6). Based on previous reports (Schmidt, Giacomelli, & Soldi, 2005; K. Wang & Arntfield, 2014), the bands were assigned to different protein secondary structures as follows: 1646-1664 cm⁻¹ corresponded to α-helix, 1615-1637 and 1682-1700 cm⁻¹ to β-sheet, 1664-1681 cm⁻¹ to β-turn, and 1637-1645 cm⁻¹ to random coils. After ACP treatment, the absorption of PPC still showed all the major secondary structure components, but the absorption intensity increased, especially for α-helix at 1658 cm⁻¹ and β-sheet at 1629 cm⁻¹. This result indicates some re-organization of the PPC secondary structures, but no obvious protein unfolding was observed by ACP treatment.

Heating of the native PPC suspension at 90° C. for 30 min only partially unfolded pea protein due to its high denaturation temperature. Heating of ACP treated PPC at 70° C. also did not lead to significant protein denaturation, but interestingly a self-standing gel was able to be formed. When heating ACP-treated PPC at 80 and 90° C., obvious protein denaturation was observed as reflected by the disappearance of the major protein secondary structural components, accompanied by significant increase of the absorptions at 1691 and 1608 cm⁻¹, suggesting pea protein aggregation after unfolding. The band at 1619 cm⁻¹ corresponded to intermolecular β-sheets caused by aggregation via hydrogen bonds (Clark, Saunderson, & Suggett, 1981), while the one at 1691 cm⁻¹ indicated antiparallel β-sheets (Byler & Susi, 1986). This result indicates that ACP treatment enabled pea protein denaturation at reduced temperature of (80-90° C.) to facilitate protein interaction development for gel formation.

### Statistical Analysis

All experiments in this section were performed in three independent batches and the data were reported as mean±standard deviations. Statistical evaluation was analyzed using a paired T-test or One-way analysis of variance followed by post-hoc test (Fisher's Least Significant Difference (LSD)). All of the analyses were conducted using SPSS 21.0 software (IBM Corp., USA) with a probability of p<0.05 considered to be significant.

### Examples Section 2—pH Shifting

### Materials and Chemicals

Pea protein concentrates (PPC 55% protein) were provided by AGT Foods and Ingredients (Saskatchewan, Canada). Reagents and chemicals used in the experiments were purchased from Sigma-Aldrich (MO, USA) and Fisher Scientific (Ontario, Canada).

### Extraction of Pea Protein Isolates (PPI)

PPC were dissolved in distilled water with the solid to solvent ratio of 1:6 to make a PPC suspension. Sodium hydroxide (NaOH, 6 mol/L) aqueous solution were added until pH 9 to further solubilize pea protein, followed by one hour of stir at room temperature (22° C.). Afterwards, the suspension was centrifuged at 8000 rpm for 15 min using high performance centrifuge (Acanti® J-E centrifuge, Beckman Coulter, USA) to obtain the supernatant, followed by acidic precipitation to pH 4.5 using 6 mol/L HCl solution and centrifugation with the same condition as above to obtain the sediment. The sediment was washed with distilled water and adjusted the pH back to 7.0 by addition of 6 mol/L NaOH solution, stirring well to form a solution. The protein solution was dialyzed using regenerated cellulose tubing (Spectra/Por® 3, 54 mm flat width, molecular weight cut-off: 3.5 kDa, Spectrum Chemical Mfg. Corp., Gardena, USA) against distilled water for 96 h. The dialyzed pea protein solution was freeze dried using Labconco Freezone 6L Console Freeze Dryer System with Stoppering Tray Dryer for 7 days to obtain the pea protein isolate (PPI). The protein content was 88.94% determined by Leco nitrogen analyzer (Leco, USA) using nitrogen-protein conversion factor of 5.96 (Fujihara, Kasuga, & Aoyagi, 2001).

### Preparation of Heat-Induced Pea Protein Gels

The PPI suspensions were prepared in phosphate buffer saline (1×PBS, pH 7.4) solvent at room temperature. 0.02% (w/v) sodium azide was added as antimicrobial agent. PPI solutions were adjusted to pH 12 using 1 mol/L NaOH solution and were held for different periods of time. The PPI solution were then titrated back to pH 7 with 1 mol/L HCl solution at room temperature while stirring to maintain a homogeneous suspension. The suspensions after pH-shifting treatment were heated at 92° C. for 1 or 2 h to form gels. Afterwards, the heated samples were cooled to room temperature and held at 4° C. overnight before analysis. The effect of protein concentration (17%, 21%), holding time at alkaline pH (1, 24 and 48 h) and heating time (1 and 2 h) on the pea protein gel properties was studied. The detailed sample preparation condition is described in Table 3, and the samples were labeled as 17% HO1-HE2, 17% HO24-HE1, 17% HO24-HE2, 17% HO48-HE2, and 21% HO48-HE2 (“HO” as holding time, “HE” as heating time, “%” as pea protein concentration). A heating temperature at 92° C. was chosen which was reported to be the pea protein denaturation temperature in the presence of NaCl (Shand et al., 2007).

TABLE 3
Definition of pea protein gel sample
name with different treatments.

| | Pea protein | Holding | Heating |
|---|---|---|---|
| | concentration | time | time |
| Sample name | (%) | (h) | (h) |
| 17% HO1-HE2 | 17 | 1 | 2 |
| 17% HO24-HE1 | 17 | 24 | 1 |
| 17% HO24-HE2 | 17 | 24 | 2 |
| 17% HO48-HE2 | 17 | 48 | 2 |
| 21% HO48-HE2 | 21 | 48 | 2 |

### Mechanical Properties

The gel samples with about 7 mm in length and 8.5 mm in diameter were analyzed by a universal testing machine (Instron® 5967, Instron Corp., MA, USA) with a 50 N load cell. The gel samples were compressed twice to 50% strain at room temperature with the crosshead speed of 1 mm/min. Textural parameters including hardness, springiness, and cohesiveness were computed by software (Blue Hill 2). Hardness is related to compressive stress and calculated as peak force (N) during the first compression cycle. Springiness indicates the ability of the gel to spring back after the deformation during the first compression cycle, which is measured by distance 2/distance 1 in the force-time curve (Ferreira, Calvinho, Cabrita, Schacht, & Gil, 2006). Cohesiveness indicates how well the gel perform against a second deformation compared to its resistance to the first deformation, which is represented by the ratio Area 2/Area 1 in the force-time curve (Ferreira et al., 2006).

FIG. 8 shows the mechanical properties of heat-induced pea protein gels pre-treated by pH shifting method, including compressive stress, hardness, springiness and cohesiveness. As expected, duration of holding time at alkaline pH positively affected the gel compressive stress. Within the same protein concentration of 17%, when the holding time was increased to 48 h, the peak compressive stress went up to 18.48±2.79 kPa, which was higher than the stress of 15.38±0.95 kPa with 24 h holding time, and 6.03±1.09 kPa with 1 h holding period. The compressive stress of the gel prepared with 48 h holding was comparable to the gelatin gels of 10% protein concentration (approximately 18 kPa) (Forte, D'amico, Charalambides, Dini, & Williams, 2015), and the gel made with 15% oat protein in combination with 0.5% carrageenan (25.95 kPa) (Nieto-Nieto, Wang, Ozimek, & Chen, 2016). The gel hardness value which indicates the maximum force required to compress the gel sample significantly increased from an average of 34.16 N to 104.78 N when holding period was increased from 1 h to 48 h. In comparison, pea protein without pH-shifting treatment was reported to form gels at pH 6.5 to 7.5 with hardness value in the range of 0.84 N to 25 N at 18-20% protein concentration (Shand et al., 2007; Taherian et al., 2011). Springiness indicates ability of the gel to recover its original form after the deforming force is removed; and cohesiveness corresponds to the strength of the gel internal bonds to hold it before rupture. The gel springiness and cohesiveness (FIG. 8c, 8d) also significantly increased with the extension of holding period (1 h to 48 h) from mean value of 0.71 to 0.79 and 0.43 to 0.60, respectively, indicating a more elastic and integrate gel structure formed as holding process continued.

Protein concentration significantly impacts the gel hardness value, which improved from 104.78 N to 155.27 N when the concentration was increased from 17 to 21%, while springiness and cohesiveness were not significantly affected. Protein concentration is believed to be a major factor affecting gel strength due to the fact that more unfolded protein molecules are available in close proximity for molecule interactions and gel network building.

The effects of heating time did not show significant difference in gel mechanical properties. It is worth noting that the springiness of 21% HO48-HE2 gel reached a mean of 0.82, which is comparable to the value of 0.86 from 10% gelatin gels (Marfil, Anhê, & Telis, 2012). Gelatin gels are known to have excellent elasticity due to the high solubility of gelatin molecules and the unique gel network structure created by rigid triple helix entangled by flexible cross-links, which are able to support large deformations. FIG. 8e shows that the pea protein gel prepared with 17% HO48-HE2 condition was able to spring back to completely recover its original height when compressed to 50% strain and released with a crosshead speed of 1 mm/min, revealing excellent elasticity of the gel. The results show that pea protein gel can be repeatedly compressed.

### Microstructure of Gel

SEM images of the 3D protein coagulum treated by pH-shifting method with different holding and heating period and the resultant gels are shown in FIG. 10. Difference in microstructure of 3D coagulum was observed with 1 h, 24 h, and 48 h holding at pH 12, respectively. With alkali treatment applied, pea protein started to aggregate (FIGS. 10a and 10c); with the extension of alkali treatment to 48 h, clear 3D coagulum started to form (FIG. 10f).

The corresponding resultant gels in FIGS. 10b, d, and g further explained the effect of alkali

holding treatment on the establishment of pea protein gel structure. It is noticeable that polymer-like porous protein networks were established in all resultant gel samples. The gel with 1 h holding has thick connective walls and large pore size of more than 7 μm (FIG. 10b). In contrast, the gel with 24 h holding has more uniform and smaller pore size of 3-5 μm (FIG. 10d), and the gel with 48 h holding exhibited a dense cross-linked structure with thin connective walls, giving the pore size of 1-2 μm (FIG. 10g). The network structure was comparable to the structure established in a gelatin-based gel (Yan et al., 2018) and a gel made of oat protein isolates with the addition of dextrin and carrageenan in the previous research (Nieto-Nieto et al., 2016). This can explain why the gel with 48 h holding treatment performed as the strongest in terms of mechanical properties because their uniform and dense crosslinked network structure which is important for formation of a strong gel. Concerning the effect of heating time on the microstructure of the gels, no notable difference was observed for the pore size of the gel structure (FIGS. 10d and 10e), suggesting that heating time had no effect on the gel network building in this case.

Globular proteins normally form particulate or filamentous gel by heating, depending on the environmental pH (Lefèvre, & Subirade, 2000). At a pH near a protein isoelectric point, heating cannot split the globular subunits, leading to the formation of spherical particles and a particulate gel structure. At a pH that deviates from the isoelectric point, a strong repulsive force causes the dissociation of subunits into monomers and induces extensive protein unfolding. The association of these unfolded protein chains results in flexible linear strands and filamentous gel or fine-stranded structure. The uniform polymer-like gel network microstructure created by methods of the present invention indicate a high level of pea protein unfolding. pH-shifting treatment at pH 12 (or acidic pH) can effectively interrupt disulfide bonds, hydrophobic interactions, and hydrogen bonds that establish the protein secondary and tertiary structures. This made the protein chain flexible and created more active sites to promote molecular interactions including hydrogen bonds and hydrophobic interactions when the protein was neutralized and heated, leading to a well-established gel network with better mechanical properties performance.

### Water Holding Capacity (WHC) and Swelling Ratio of Gel

The gel water holding capacity was determined following the method from Kocher and Foegeding (1993) with modifications. Approximately 0.5 gram of the gel sample was loaded in ultrafiltration tube from Vivaspin Turbo 15, 3,000 MWCO (Sartorius Stedlm Lab Ltd., Stonehouse, UK) and centrifuged at 4000 rpm for 10 min at room temperature. The separated water was discarded and the weights of the tube together with the sample gel before and after centrifugation were recorded as W1 and W2. The weight of ultrafiltration tube was recorded as W. WHC was calculated as equation (1):

$$WHC \% = \frac{W 2 - W}{W 1 - W} \times 100 \% \qquad ( 1 )$$

Approximately 0.5 gram of gel samples were soaked into 30 mL of water for up to 24 h at room temperature. Swelling ratio of pea protein gels was determined at different time point by equation (2):

$$SR \% = \frac{Ms}{Mo} \times 100 \% \qquad ( 2 )$$

Where Mo is the weight of original gel before soaking into water, and Ms as the weight of swollen gel after soaking into water.

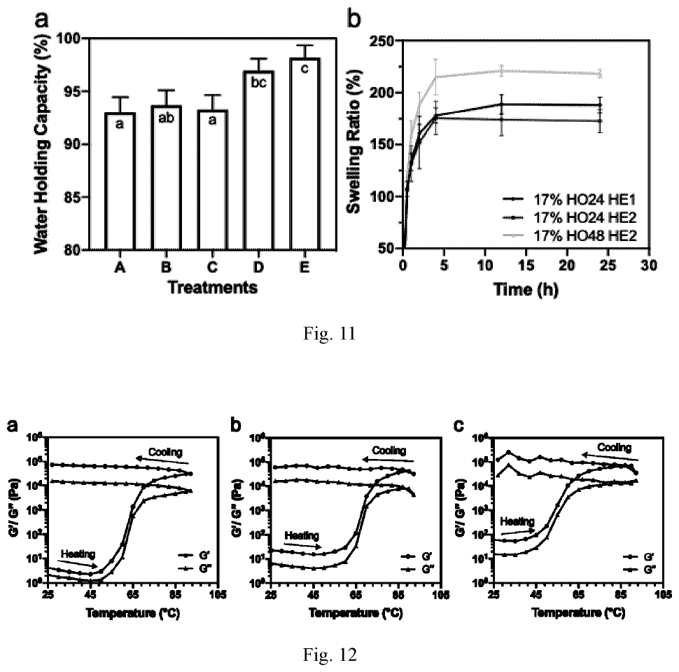

As shown in FIG. 11a, water holding capacity (WHC) of all gel samples formed by pH shifting were over 90%. Longer holding time (48 h) treated protein formed gels with higher WHC over 95% regardless of the protein concentration. In comparison, in a pea protein gel prepared with 0.3 M NaCl at pH 7 (15% w/v protein content) (Munialo et al. (2014)), syneresis was observed and the gel was not applicable for the measurement of WHC.

The pea protein gels exhibited considerably higher or comparable WHC value to the protein gels with good water holding properties, for example, the gel prepared from egg white at neutral pH had a WHC value of 90.03% (Khemakhem, Attia, & Ayadi, 2019); soy protein gels were reported to show a WHC range of 60-95% based on different preparation methods (Maltais, Remondetto, Gonzalez, & Subirade, 2005; Zhou et al., 2019). Such high WHC observed in this work was likely attributed to the uniform and dense crosslinked gel network structure achieved from the pea proteins after pH-shifting treatment. It is suggested that a gel with evenly distributed network structure tends to have better WHC as water is more tightly trapped in the dense structure (Munialo et al., 2014). Higher WHC was also found in finer-stranded network compared to thick-stranded network for the gel prepared from whey protein isolates (He, Azuma, & Yang, 2010).

FIG. 11b shows the swelling behavior of the pea protein gel prepared with different holding and heating conditions. Swelling ratio was not calculated for the 17% HO1-HE2 sample because the gel went through disruption at the 2 h time point. Time to reach swelling equilibrium was about 12 h, giving the swelling ratio of 174%, 188%, and 220% for 17% HO24-HE1, 17% HO24-HE2, and 17% HO48-HE2 gel, respectively. No significant difference in the swelling ratio was observed between the 17% HO24-HE1 and 17% HO24-HE2 sample, indicating that heating time had no influence on the swelling ratio of the gel, which is consistent with the results of mechanical properties and WHC. On the other hand, swelling ratio increased with the holding time extended from 24 h to 48 h, suggesting that the gel with more uniform and stronger network structure was able to absorb and hold more water, giving a higher swelling ratio accordingly. The above data indicate good water holding capacity and elevated swelling ratio of the pea protein gels were due to their unique highly crosslinked polymer-like gel microstructures after alkaline pH-shifting treatment (FIG. 10). High WHC and swelling ratio of pea protein gel can be desirable since it can be used in food formulations such as baked products, meat and dairy products to modify the texture through moist binding as well as improve the quality of food products by preventing moist loss.

### Rheological Properties of Pea Protein Gels

The formed gels were tested on a DHR-3 rheometer (TA Instruments, DE, USA) to evaluate their viscoelastic properties. Samples were loaded on 40 mm parallel plate geometry with a gap of 1 mm. The value of the strain amplitude was set as 1%. The frequency sweep measurements were performed at 25° C. at an angular frequency (w) from 0.1 to 100 rad/s. Lines of storage modulus (G′) and loss modulus (G″) in response to frequency were plotted.

In order to monitor the change of rheological properties during the formation of pea protein gels, the protein suspensions after pH shifting back to pH 7 were tested during temperature ramps at a rate of 5° C./min from 25 to 92° C. and then cooled to 25° C. at a rate of 5° C./min. Parallel plate geometry (40 mm) with a gap of 1 mm was used to measure viscoelastic parameters including storage modulus G′ and loss modulus G″. Approximately 1.5 mL of each suspension sample was loaded for the dynamic temperatures sweep measurements and the angular frequency (w) was set to be 1 Hz.

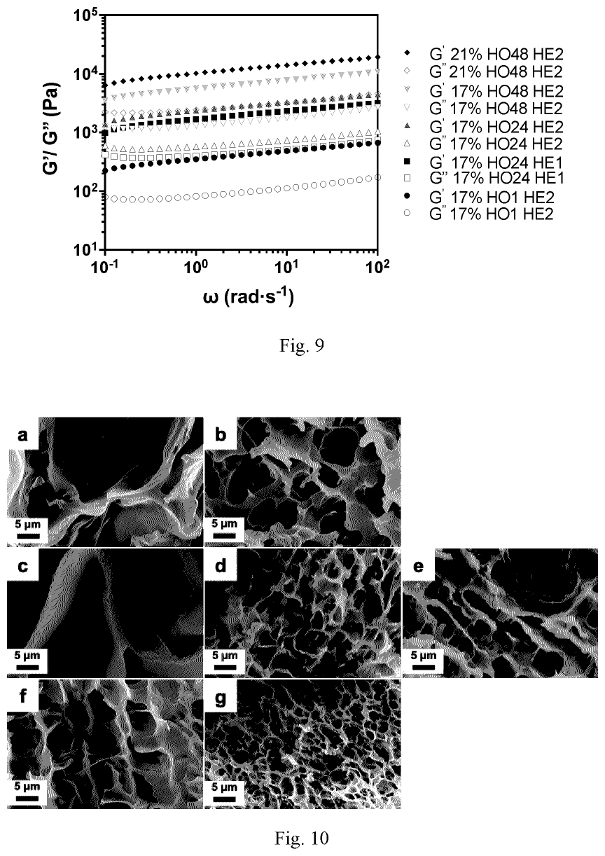

The rheological behavior of the resultant gels is shown in FIG. 9. The storage modulus (G′) of all gel samples was higher than the loss modulus (G″) value, indicating elasticity dominant in the gel. The frequency sweep measurements give information of gel networks linked through covalent bonds or non-covalent bonds and similar patterns concerning G′ and G″ were observed in all gel samples. G′ values showed relative low frequency dependence, which was comparable to the gel prepared from egg albumin (Tunick, 2011), showing that non-covalent bonds including hydrophobic interactions and hydrogen bonds might be controlling the gel network. This further confirms that strong gels were formed from pea protein after pH-shifting treatment. Among different treatments, holding time variation exhibited a more noticeable effect on the viscoelastic properties of the gels compared to the variables of heating time and protein concentration as a more significant change of G′ and G″ value was observed in gels with different holding time.

Temperature dependence of the gel rheological properties was shown in FIG. 12 to observe the behavior upon heating during the gel formation. It is noticeable that G′ was higher than G″ initially before heating, indicating elastic behavior outweighed the viscous behavior after the pH shifting treatment. This is in agreement with the microstructure observed for the pH-shifting treated 3D pea protein coagulum samples where crosslinking structure started to form already. Damodaran (2008) suggested that hydrophobic interactions occur and dominate at high temperature as they are endothermic, so the molecular forces occurred after the sharp increase in G′ value could be induced by hydrophobic interactions.

Sharp increase in both G′ and G″ occurred at different temperature for samples treated by different holding time, with 1 h and 24 h holding sample at approximately 65° C. (FIGS. 5a and 5b), whereas at 55° C. for 48h holding sample (FIG. 5c). For comparison, pea protein isolates without pH shifting treatments in the previous research (Sun & Arntfield, 2010) showed sharp increase of G′ and G″ value at 80° C. This result suggests that pea protein was partially unfolded by pH-shifting and then was further developed into three-dimensional gels by heating treatment starting at 55-65° C. Higher level of protein unfolding for samples with longer holding time during pH-shifting treatment may explain their lower gelling temperature and the higher G′ value for the resultant gels (FIG. 5c). The gel development at reduced temperature may benefit pea protein applications as a functional ingredient. For example, when the pH-shifted pea protein is applied as a gelling or thickening agent in meat products, a reduced heating temperature will improve overall quality by producing less cooking loss and increasing juiciness.

It is noticeable that the G′ value barely went up during the cooling procedure in all treatments, which suggests that heating process played much more important roles in establishing the formation of protein crosslinks than the cooling process did.

### Pea Protein Gel Morphology

Protein suspensions after pH-shifting treatment and the formed gels were frozen in liquid nitrogen, and then freeze-dried. Cross-sectional view of the freeze-dried 3D pea protein coagulum and gels were sputter-coated with gold and the morphology was observed using a Philips XL-30 scanning electron microscope (SEM) at an acceleration voltage of 20 kV.

### Protein Conformational Change

Pea protein was analyzed by FTIR (Fourier Transform Infrared Spectroscopy) at different steps in both dried and wet forms during the gel preparation to understand the protein conformational change. Pea protein isolate powder and the freeze dried pea protein gel samples were analyzed in KBr plate with KBr to sample ratio as 1:100 in weight and the spectra was recorded by Nicolet 6700 spectrophotometer (Thermal Fisher Scientific Inc., Pittsburgh, PA, USA) along the wavenumber from 400 to 4000 cm⁻¹ with 128 scans at a 4 cm⁻¹ resolution. Wet samples including 3D coagulum at pH 12 and the resultant gel samples were prepared with D₂O, 1M DC1, and 1M NaOD. The wet samples were analyzed between CaF₂ windows separated by a 25 mm polyethylene terephthalate film spacer with the spectra recorded at the same condition as previously described. Fourier self-deconvolution for amide I region was analyzed using Omnic 8.1 software at a bandwidth of 24 cm⁻¹ and an enhancement factor of 2.5.

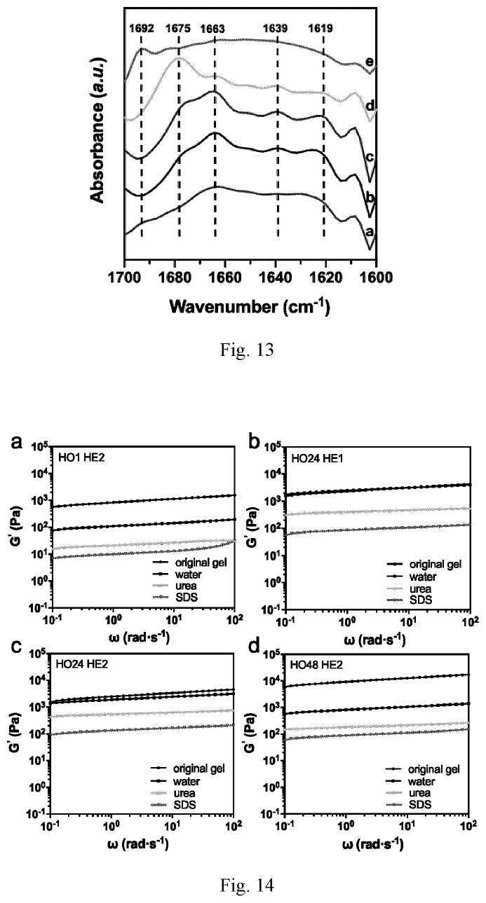

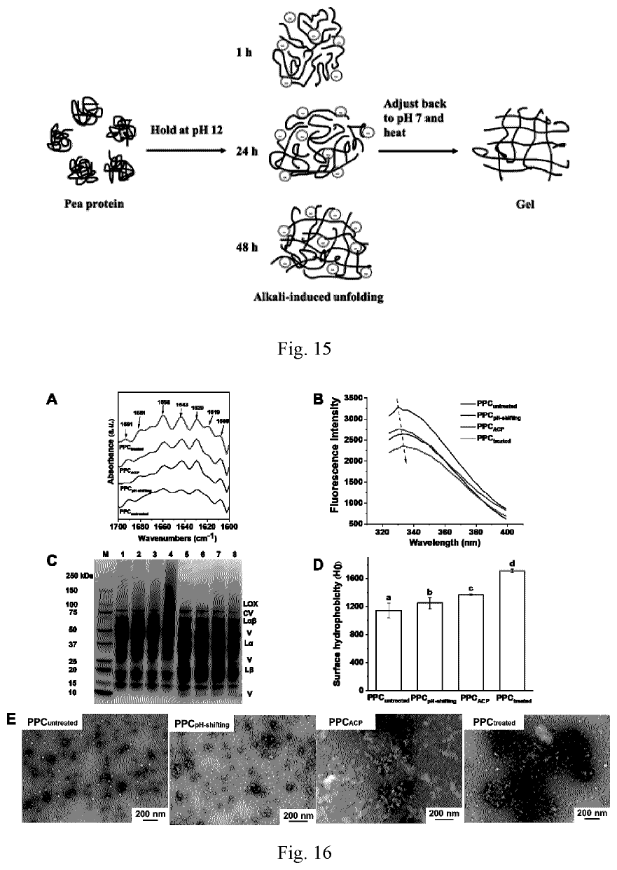

FTIR spectra results are displayed in FIG. 13 to show the band of amide I region. The characterization of spectrum was referenced from Byler and Susi (1986) as: 1619 and 1692 cm⁻¹ (β-sheets), 1651 and 1663 cm⁻¹ (α-helix), and 1639 cm⁻¹ (random coil). After pH-shifting treatment (FIGS. 13b, c and d), two new peaks appeared at 1619 and 1675 cm⁻¹, suggesting pea protein unfolding and aggregation occurred during pH-shifting treatment. The band of 1619 cm⁻¹ corresponded to intermolecular β-sheets caused by aggregation via hydrogen bonds, while the one at 1675cm⁻¹ indicated the antiparallel β-sheets (Byler & Susi, 1986). With the increase of the holding time at alkaline pH, the absorption intensity at 1619 and 1675 cm⁻¹ increased, accompanied by the decrease of the α-helix structure at 1663 cm⁻¹, confirming increased level of protein unfolding and aggregation when hold time was prolonged. Such increased protein crosslinking was also confirmed by the SEM photo for the 3D protein coagulum treated with 24 h and 48 h of holding time. Ma, Rout, Chan, and Phillips (2000) suggest that the unfolding of the protein upon pH treatment is likely due to the disruption of hydrogen and hydrophobic interaction, which is induced by the high charge repulsion. The exposure of the hidden active groups then facilitated protein aggregation through formation of new interactions including the hydrogen bonds that stabilize the intermolecular β-sheets in the aggregates. After heating treatment to induce gel formation, the peak at 1692 cm⁻¹ re-appeared and dominated as shown in FIG. 6e, whereas other protein secondary structures almost disappeared, suggesting full pea protein unfolding to promote gel network formation during heating process.

### Molecular Interactions in Pea Protein Gels—Gel Dissociation Test

In order to understand the molecular interactions involved in the formation of pea protein gels, the gel samples prepared with different holding and heating time were soaked into water (controls), 2M urea, 0.02M 2-mercaptoethanol (2-Me), and 1 wt % sodium dodecyl sulfate (SDS) solution as dissociation reagents for 48 h to disrupt hydrogen bond, disulfide bond, and hydrophobic interactions, respectively. Frequency sweep analysis were performed on the soaked gel samples over a range of 0.1-100 rad/s at 25° C. with 1% strain. Storage modulus (G′) in response to frequency were plotted and shown in FIG. 14.

The gels that immersed in 2-Me became too rigid to be tested, which might due to the replacement of water in the gel by 2-Me solvent. This also suggests that disulfide bonds might not be involved in the formation of the pea protein gel. This is probably owing to the low cysteine content in pea proteins. A very low amount of cysteine content as 0.87% was reported for pea protein in the study from Pownall, Udenigwe, and Aluko (2010). In the gel with 48 h holding time during pH-shifting treatment (FIG. 14d), hydrophobic interactions and hydrogen bonds were of similar importance as the corresponding G′ value reduced to similar extent when immersed in SDS and urea solution. Different patterns were observed in the gel with 24 h holding time, in which the gel immersed in SDS was more affected as reflected by lower G′ value than the gel immersed in urea (FIGS. 14b and 14c), indicating that the hydrophobic interactions were the dominating forces compared to hydrogen bonds. The result suggests that hydrophobic interactions and hydrogen bonds were both involved in the gel formation of pea protein after pH-shifting treatment and longer holding time at alkaline pH favored hydrogen bonding formation to strengthen the gels, while no evidence shows that disulfide bonds are required for the gelation.

### Statistical Analysis

All experiments in this section were performed in three independent batches and the data were reported as mean±standard deviations. Analysis of variance (ANOVA) was performed to analyze the effects of holding time, heating time, and protein concentration on mechanical properties of gels, followed by Tukey's test to compare multiple means. A probability of p<0.05 was considered to be statistically significant.

### Examples Section 3—Combined ACP and pH Shifting

Pea protein flours were provided by AGT Foods Research & Innovation Centre (Saskatoon, SK, Canada), which contained protein (53.1%), carbohydrate (33.7%), fat (2.0%), ash (5.3%), and moisture (5.9%) (S. Zhang et al., 2020). Broad-range SDS-PAGE molecular weight standards, SDS-PAGE 4-15% gels (Mini-PROTEAN® TGX™), sample buffer, and running buffer were purchased from Bio-Rad Laboratories Inc. (Hercules, CA, USA). Ethylenediaminetetraacetic acid (EDTA), Trizma® base, sodium dodecyl sulfate (SDS), 5,5′-dithiobis-(2-nitrobenzoic acid) (DTNB), 2,4,6-trinitrobenzene sulfonic acid (TNBS) (5% (w/v) in H₂O), 2-mercaptoethanol (2-ME), 1-anilinonaphthalene-8-sulfonic acid (ANS), L-lysine, and Bradford Reagent were purchased from Sigma-Aldrich (St. Louis, MO, USA). Urea, bovine serum albumin (BSA), and other chemicals (analytical grade) were purchased from Fisher Scientific (Ottawa, ON, Canada).

Atmospheric Cold Plasma (ACP) and pH-Shifting Pre-Treatment

Pea protein concentrate (PPC) was extracted from the pea protein flour by the method described previously (J. Yang, Liu, Zeng, & Chen, 2018) with slightly modification. Briefly, the pea protein flour was dispersed into water at a flour-to-water ratio of 1:7. The mixture was adjusted to pH 9 and stirred for 1 h. After centrifuged at 8000 g for 15 min, the supernatant was adjusted to pH 4.5, followed by centrifugation to precipitate the proteins. Finally, the protein precipitate was neutralized and dialysis to remove salt. After freeze-drying, the PPC was stored in 4° C. before analysis. The obtained PPC has a protein content of 85.6% as determined by a nitrogen-to-protein conversion factor of 5.96 (Fujihara, Kasuga, & Aoyagi, 2001) using a Leco nitrogen analyzer (Leco®, USA). PPC suspension in distilled water (12 wt. %, pH 7.0) was stirred at 350 rpm overnight at 4° C. After raising to room temperature (22-23° C.), the PPC suspension was adjusted to pH 12 with 1 M NaOH. 2.5 g of PPC suspension (12 wt. %) was transferred to a plastic container (4 cm in diameter and 0.5 cm in height, METER®, Pullman, WA, USA). Then the plastic container was placed between a high-voltage electrode and a ground electrode for treatment by a dielectric barrier discharge plasma system (PG 100-D, Advanced plasma solutions, Malvern, USA). The processing conditions were set up as follows: frequency of 3500 Hz, the duty cycle of 70%, the pulse width of 10 μs, the distance from the sample surface to the high-voltage electrode of 2 mm, the current output of 0-1 A, the voltage output of 0-30 kV, and a total treatment time of 10 min (2 min/time, 5 times). The temperature of ACP-treated samples increased from around 21.0° C. to 24.0° C. The system pH decreased from 12 to around 11.5. Immediately after the ACP treatment, the pH was adjusted back to 7.0 with 1 M HCl. Finally, the treated dispersions were spray-dried to obtain the modified pea protein powder labeled as PPC_{treated}. The control samples were protein powder treated with pH-shifting alone labeled as PPC_{pH-shifting}, and protein powder treated with ACP alone labeled as PPC_{ACP}, as well as protein sample without any treatments labeled as PPC_{untreated}.

Key parameters of ACP including voltage output (0-15 and 0-30 kV), frequency (2000 and 3500 Hz), and treatment time (2-15 min) were tested. From there, we identified 0-30 kV of voltage output, 3500 Hz of frequency, and over 10 min of treatment time were preferred as allowing formation of stronger pea protein gels. Longer exposure time was not preferred for economic reasons. Thus, the following conditions (3500 Hz, 10 μs, 10 min, 0-30 kV, and 0-1 A) were applied.

### Characterizations of the Modified PPC Powder

### Protein Morphology and Conformation

Transmission electron microscopy (TEM) was used to analyze the morphology of the modified PPC powders. A drop of PPC_{untreated}, PPC_{pH-shifting}, PPC_{ACP}, and PPC_{treated} suspensions of 0.05 wt. % was deposited on carbon film-covered 400 mesh copper grids and allowed to settle for one minute, respectively. Excess was removed with filter paper. Next, the samples were negatively stained with 4% uranyl acetate for several seconds, and then the excess was wicked away. After sufficient drying, grids were examined at 80 kV on a transmission electron microscope (TEM, Morgagni 268, Philips-FEI, Hillsboro, USA).

Morphology of the PPC_{untreated}, PPC_{pH-shifting}, PPC_{ACP}, and PPC_{treated} suspensions are presented in FIG. 16E. The TEM images of PPC_{untreated} and PPC_{pH-shifting} present the spherical particles with some level of aggregation. On the other hand, aggregation was obvious for PPC_{ACP} and PPC_{treated} According to analyzing the optical emission spectra of atmospheric air DBD plasma (Feizollahi, Arshad, Yadav, Ullah, & Roopesh, 2020), ACP discharge was dominated by excited nitrogen second positive system and nitrogen first negative system, followed by OH radicals. These excited nitrogen, atomic oxygen, and OH radicals could transfer into the liquid and then could be easily converted to other ROS and RNS, such as H₂O₂, NO₂, and NO₃⁻ (Zhou et al., 2018). These radicals could promote the oxidation of sulfhydryl groups and the subsequent formation of disulfide bonds between cysteine moieties. This may explain the formation of obvious aggregates by ACP treatment. When ACP treatment was combined with pH-shifting, the aggregates became larger and more connected. This was probably caused by partial protein unfolding by the combined treatment that exposed more active groups to promote aggregate growth.

Protein conformation before and after treatment was characterized by Fourier transform infrared spectroscopy (FTIR) and intrinsic fluorescence spectroscopy. FTIR spectra of samples were recorded by a Nicolet 6700 Fourier transform infrared spectrophotometer (Thermo Fisher Scientific Inc., MA, USA). All untreated and treated PPC powder samples were dissolved in D₂O to prepare suspensions and gels, which were placed between two CaF₂ windows separated by a 25 μm polyethylene terephthalate film spacer for measurement. The sample was tested from 4000 to 1111 cm⁻¹ under 128 scans at 4 cm⁻¹ resolutions. The background was the spectrum of air tested at the same condition and subtracted automatically. Fourier self-deconvolution (FSD) on amide I band (1700 -1600 cm⁻¹) in each spectrum with bandwidth at 24 cm⁻¹ and enhancement factor at 2.5 was analyzed with Omnic 8.1 software (Thermo Fisher Scientific, MA, USA) to evaluate the secondary structures (T. Lefèvre & Subirade, 1999). The intrinsic fluorescence spectra of protein suspensions were determined using a SpectraMax M3 microplate reader (Molecular Devices, USA). The samples were diluted with distilled water to 2.5 mg/mL before testing. Emission spectra between 300 to 400 nm were recorded at an excitation wavelength of 295 nm. The slit width was set to 3 nm for both excitation and emission, and the scanning speed was 10 nm/s.

The protein tertiary structural changes can be reflected by the intrinsic fluorescence spectrum of tryptophan because it is sensitive to the polarity of microenvironments. All samples were diluted to 2.5 mg/mL with distilled water before testing. FIG. 16B shows that the fluorescence emission of PPC_{untreated} and PPC_{ACP} at 330 nm. The cold plasma treatment alone led to a decrease in the fluorescence spectra of pea protein. A decrease in the fluorescence intensity was also observed for pH-shifting accompanied by a red shift to 333 nm. When ACP was combined with pH-shifting, a red shift to 333 nm was also observed together with a higher level of fluorescence intensity decrease. This result suggests protein conformational changes, possibly tryptophan residues moved from a hydrophobic microenvironment towards a more hydrophilic microenvironment. A greater change in the tryptophan environment and protein conformation was observed by the combined treatment (Miriani, Iametti, Bonomi, & Corredig, 2012). The conformational changes of whey protein, (W. Chen et al., 2019) soy protein (J. Jiang, Chen, & Xiong, 2009), egg white protein (Yu et al., 2021), and peanut protein (Y. Wang et al., 2020) treated by pH-shifting treatment also showed a red-shift in the fluorescence spectra or combined with a decrease in the fluorescence intensity, partially attributed to the interruption of hydrogen bonding under alkaline conditions. The fluorescence and FTIR results together suggest that both ACP and pH-shifting triggered protein conformational change, which mainly occurred at the protein tertiary level because ACP treatment was short (10 min), and the pH was shifted back to neutral immediately after the treatment. The greater conformational change was observed by the combined treatment that led to protein aggregation as observed by FTIR.

The absorption bands of deconvoluted FTIR spectra of the amide I band (1600-1700 cm⁻¹) can be assigned as follows: 1608 cm⁻¹ (vibration of amino acid residues), 1629 cm⁻¹(β-sheet), 1643 cm⁻¹ (unordered structures), 1658 cm⁻¹ (α-helix), 1691 cm⁻¹ (β-sheet). When the sample was only treated by pH-shifting, the PPC was exposed to pH 12 for 10 min, then adjusted back to pH 7 followed by spray-drying. When the sample was treated only by ACP, the protein concentrate was treated by ACP at neutral pH for 10 min followed by spray-drying. The PPC_{pH-shifting} and PPC_{ACP} showed similar absorptions in FTIR when compared to PPC_{untreated}. While for the pea protein concentrates by combined ACP and pH-shifting treatment, two new peaks appeared at 1619 and 1681 cm⁻¹ in the spectrum of PPC_{treated}. (Ellepola, Choi, & Ma, 2005) The band at 1619 cm⁻¹ is assigned to intermolecular β-sheets via hydrogen bonds resulting from aggregation, while the band of 1681 cm⁻¹ indicates that the β-sheets are antiparallel. Considering the α-helix at 1658 cm⁻¹ and β-sheet at 1629 cm⁻¹ were still obvious after the treatment, the result suggests that the combined ACP and pH-shifting treatment triggered partial unfolding of pea protein and aggregation.

Zeta potential—The zeta potential measurement of PPC_{untreated}, PPC_{pH-shifting}, PPC_{ACP}, and PPC_{treated} suspensions (1 mg/mL) at neutral pH was carried out on a Zetasizer Nano-ZS (Malvern Instruments Ltd., UK) at 25° C. The particle (pea protein) and dispersant (distilled water) refractive index (RI) was set to 1.45 and 1.33, respectively, and the absorption index was 0.001 (R. Wang, Tian, & Chen, 2011).

The zeta-potential values are summarized in Table 4. After pH-shifting or combined ACP and pH-shifting treatment, the pH of the protein suspensions was adjusted back immediately, thus the four samples were measured at the neutral pH. PPC_{untreated}, PPC_{pH-shifting}, PPC_{ACP}, and PPC_{treated} were all negatively charged due to the deprotonation of the —COO⁻ group at neutral pH. Protein colloids with a high absolute value of the zeta potential (˜30 mV) are electrically stabilized (Klost, Brzeski, & Drusch, 2020). The absolute values of PPC_{untreated} (−33.80 mV), PPC_{pH-shifting} (−31.73 mV), PPC_{ACP} (-31.03 mV), and PPC_{treated} (−31.50 mV) samples were above 30 mV, suggesting that pea protein with or without treatments were not susceptible to coagulate or flocculate.

TABLE 1
Zeta potential, —NH₂ contents, and —SH contents of pea protein
concentrate (PPC) suspensions (PPC_{untreated}) treated by pH-shifting alone
(PPC_{pH-shifting}), atmospheric cold plasma (ACP) treatment alone (PPC_{ACP})
or combined ACP and pH-shifting treatment (PPC_{treated}).

| | | —NH₂ | —SH |
|---|---|---|---|
| | Zeta | contents | contents |
| | potential | (μmol —NH₂/mg | (μmol/g |
| Samples | (mV) | protein) | protein) |
| PPC_{untreated} | −33.80 ± 0.56^{b} | 1.25 ± 0.20^{a} | 19.46 ± 0.19^{d} |
| PPC_{pH-shifting} | −31.73 ± 0.32^{a} | 1.24 ± 0.07^{a} | 14.05 ± 0.27^{b} |
| PPC_{ACP} | −31.03 ± 0.85^{a} | 1.24 ± 0.05^{a} | 17.75 ± 0.49^{c} |
| PPC_{treated} | −31.50 ± 0.36^{a} | 1.24 ± 0.07^{a} | 9.35 ± 0.12^{a} |

Different lower-case letters within a column denote significant differences (p < 0.05), as determined by One-way analysis of variance (ANOVA) followed by a post-hoc test (Fisher's Least Significant Difference (LSD)).

### SDS-PAGE

Sodium dodecyl sulphate-polyacrylamide gel electrophoresis (SDS-PAGE) was performed under non-reducing (NR) and reducing (R) conditions (Chihi, Mession, Sok, & Saurel, 2016). Protein samples (5 mg/mL) were mixed with the 2×sample buffer (Bio-Rad Laboratories Inc.) with (R) or without (NR) 5% 2-mercaptoethanol (2-ME). Then the mixtures were boiled for 5 min and cooled to room temperature. All the sample mixtures were centrifuged at 10,000×g for 5 min before electrophoresis. 12 μL of supernatants and 5 μL of Broad-range SDS-PAGE molecular weight standards (Bio-Rad Laboratories, Hercules, CA) were loaded onto SDS-PAGE 4-15% gels (Mini-PROTEAN® TGX™, Bio-Rad Laboratories Inc.), respectively. After the running of protein lanes at a constant voltage of 80 V, the gel was stained for 20 min using 0.1% (w/v) Coomassie brilliant blue-R250 in methanol/ acetic acid/water (2:9:9, v/v/v) and destained in methanol/ acetic acid/ water (1:1:8, v/v/v) overnight.

The SDS-Page patterns of the PPC_{untreated}, PPC_{pH-shifting}, PPC_{ACP}, and PPC_{treated} were obtained under non-reducing (NR, line 1-4) and reducing conditions (R, line 5-8) with the former conducted in the presence of SDS, and the latter with both SDS and 2-ME (FIG. 16C). Sodium dodecyl sulfate (SDS) and 2-mercaptoethanol (2-ME) cleave physical interactions and disulfide bonds in proteins, respectively. The electrophoresis patterns showed characteristic bands of pea protein subunits, including lipoxygenase (LOX) (˜100 kDa), convicilin (CV) (˜70 kDa), legumin (Lαβ) (˜60 kDa), and vicilin (V) (48-52 kDa). Legumin subunits contained acidic (Lα, ˜40 kDa) and basic (Lβ, ˜20 kDa) polypeptides. Vicilin subunits can be dissociated into fragments with low molecular weight (12-16, 20, and 25-36 kDa). There was no significant difference between PPC_{untreated} and PPC_{pH-shifting} under either the non-reducing or the reducing conditions, implying that alkaline treatment alone for 10 min was too short to impact the pea proteins subunits. However, with regard to the PPC_{treated} sample, the pattern in lane 4 shows the formation of aggregates of pea protein between 100 kDa and 150 kDa after combined ACP and pH-shifting treatment in FIG. 16B. Meanwhile, under the non-reducing condition (line 4), the intensity in the Lαβ band and vicilin band of ˜50-75 kDa decreased, demonstrating the aggregates of 100-150 kDa were composed with both legumin subunits and vicilin. This band of molecular weight of 100-150 kDa was diminished in the pattern under reducing conditions (line 8), which means that disulfide interactions played an important role in the formation of the PPC aggregates. In addition, PPC_{pH-shifting}, PPC_{ACP}, and PPC_{treated} showed no difference in their electrophoresis profiles under reducing conditions in comparison to the PPC_{untreated}, indicating that pH shifting alone, ACP treatment alone, and their combination did not significantly change the pea protein subunits. However, the formation of large molecule aggregates of pea protein treated by pH 12-shifting for 1 h was revealed through the protein profile by Jiang et al. (2017). The different results might be due to the short pH-shifting time (5 min) in this study. In addition, it was reported that ACP alone did not modify the peanut protein primary structures in previous research studies (Ji et al., 2018). To the best of our knowledge, this is the first research that characterized pea protein structures by combined ACP and pH-shifting treatment. Future research is required to understand its impact on pea protein primary structures such as chemical modifications of the amino acid side chains.

### Surface Hydrophobicity (H₀)

Surface hydrophobicity (H₀) was measured by using the 1-anilinonaphthalene-8-sulfonic acid (ANS) as a fluorescent probe, according to the method of Haskard (Haskard & Li-Chan, 1998) with some modification. PPC_{untreated}, PPC_{pH-shifting}, PPC_{ACP} and PPC_{treated} suspensions (0.005-1.25 mg/mL) were prepared in 0.1 M phosphate buffer (pH 7.4). 20 μL of 8 mM ANS solution was blended with 4 mL of each dilution, followed by keeping in dark for 15 min. The fluorescent intensity was analyzed at 460 nm (emission) with the excitation wavelength of 390 nm using a SpectraMax M3 microplate reader (Molecular Devices, USA) with a slit width of 3 nm. The initial slope of the corrected fluorescence intensity against the protein concentration plot was used as an index of surface hydrophobicity.

Both pH-shifting and ACP treatment alone slightly increased the protein surface hydrophobicity (H₀) value from 1141 to 1249 and 1366, respectively. A greater level of H o increase (H₀ value 1710) was observed when the pea protein was treated by combined ACP and pH-shifting (FIG. 16D), suggesting that more nonpolar amino acid residues were exposed to the surface due to partial protein unfolding and many of these nonpolar residues were still maintained at the surface even after the protein aggregation. Similarly, an increased H₀ were observed in pea protein (Jiang et al., 2017), soy protein (Yildiz, Andrade, Engeseth, & Feng, 2017) and peanut protein (Li et al., 2020) after pH-shifting at 12 treatments alone. Mehr & Koocheki (2021) found that the H₀ of Grass pea protein isolate significantly increased with increasing the ACP treatment time and voltage.

### Protein Solubility

Soluble protein content was quantified by the Bradford method (Bradford, 1976). A standard curve was established using bovine serum albumin (BSA) (0.1-1.6 mg/mL). The PPC_{untreated}, PPC_{pH-shifting} and PPC_{treated} powders (0.5 g) were dispersed in 10 ml water, and then the suspensions were centrifuged at 10,000×g for 10 min at 4° C. 4 μL of the diluted sample (1 mg/mL) was mixed with 200 μL of Bradford Reagent in a 96-well clear polystyrene plate (Costar #9017, Corning, Inc., NY). Blank used distilled water instead of sample. After 20 min, the absorption value of the supernatant was measured at 595 nm using a SpectraMax M3 microplate reader (Molecular Devices, USA). The protein solubility was calculated by Equation (2):

$$Protein solubility ( \% ) = \frac{Protein content of supernatant ( mg mL )}{Total protein content ( mg mL )} \times 100 \% \qquad ( 2 )$$

### Free Sulfhydryl Group (—SH) and Amino Group (—NH₂) Contents Assay

Free —SH group levels were analyzed according to the method of Ji et al. (Ji et al., 2018). 0.5 mL of PPC_{untreated}, PPC_{pH-shifting}, PPC_{ACP}, and PPC_{treated} suspensions (5 mg/mL) were mixed with 2.5 mL of Tris-Gly buffer with 8 M urea and 0.02 mL of Ellman's reagent (4 mg/mL). Blank used corresponding Tris-Gly buffer with 8 M urea. The mixture was then allowed to stand in dark for 1 h at 25° C., and the absorbance was recorded at 412 nm using a SpectraMax M3 microplate reader (Molecular Devices, USA). The —SH group contents were calculated by Equation (3).

$$SH ( \frac{µmol}{g} ) = \frac{75.53 \times {A}_{412} \times D}{C} \qquad ( 3 )$$

Where A₄₁₂ is the absorbance at 412 nm, D is the dilution factor, and C is the sample concentration (mg/mL).

The free —NH₂ content was analyzed by the trinitrobenzene sulphonate (TNBS) method (Habeeb, 1966). 2 mL of the PPC_{untreated}, PPC_{pH-shifting}, PPC_{ACP}, and PPC_{treated} solutions (0.2 mg/mL) in 0.1 M sodium bicarbonate was mixed with 1 mL of TNBS solution (0.01% (w/v) and the mixture was kept for 2 h at 37° C. After termination of the reaction by 0.5 mL of 1 M HCl, the absorbance of the mixture was recorded at 335 nm using a SpectraMax M3 microplate reader (Molecular Devices, USA). Serial dilutions of L-lysine (2.5, 5, 10, 20, and 40 μg/mL) instead of sample solutions were prepared to plot a standard curve.

The changes in —NH₂ content of proteins can be considered as an indicator of the degree of hydrolysis. There was no significant difference in —NH₂ content in the four samples (Table 1), indicating there was no obvious pea protein hydrolysis by pH-shifting or ACP treatment alone or their combination treatment. Meanwhile, the free —SH contents greatly decreased after combined ACP and pH-shifting treatment. This result is in accordance with the SDS-Page, suggesting that the disulfide bonds played an important role in pea protein aggregation and growth promoted by free radicals generated by ACP when the protein structure was partially unfolded. Similarly, ACP treatment decreased the free —SH groups in Grass pea protein isolate because of the high sensitivity of sulfur-containing amino acid side chains to oxidation and the conversion of —SH groups to disulfide bonds (Mehr & Koocheki, 2021). In contrary, an increase in the free —SH groups after pH 12-shifting for over 1 h was observed in ginkgo seed protein (Zhang et al., 2021), soy protein isolate (Jiang et al., 2009), and peanut protein isolate (Li et al., 2020) due to the exposure of internal —SH groups and breakage of disulfide bonds.

### Viscosity

The viscosity of PPC_{untreated}, PPC_{pH-shifting}, and PPC_{treated} suspensions (14 wt. %) was measured using a DHR-3 rheometer (TA Instruments, New Castle, DE, USA) with a 40 mm diameter and angle of 1.997° cone plate geometry. The sample (about 1.5 mL) was loaded onto the center of the parallel plate and the gap between plates was set to 1 mm. The sample was equilibrated for 5 min before testing to obtain a desirable temperature (25° C.). The flow measurement was performed over a shear rate range of 0.1-500 s⁻¹, and the viscosity (11) curve was obtained from the data analysis software.

### Re-Hydration and Viscosity

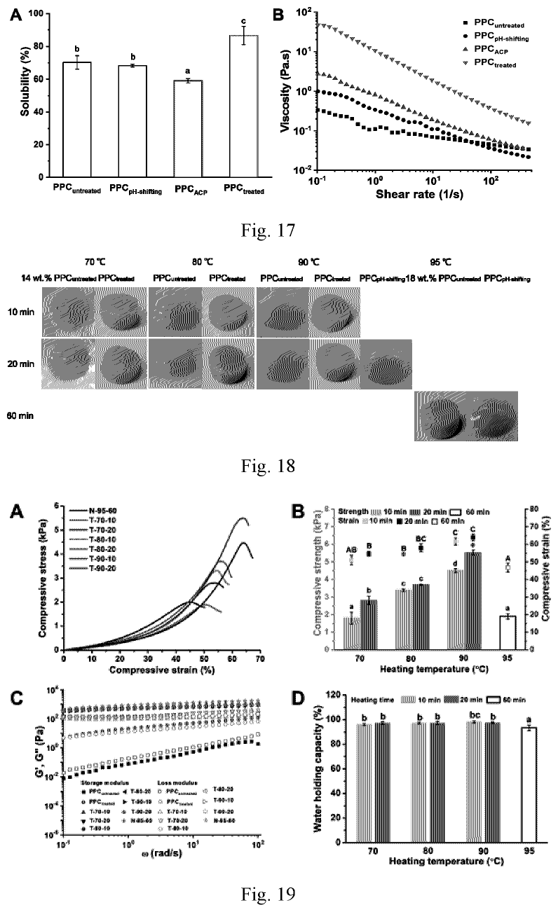

Protein powder rehydration ability is one of the most important physicochemical properties to enable good functional properties of protein ingredients for their wide applications. The PPC_{untreated} and PPC_{pH-shifting} showed solubility of ˜70%. (FIG. 17A). The solubility of PPC_{ACP} was decreased to ˜60%, possibly due to the formation of insoluble protein aggregates by ACP treatment. Interestingly, the solubility was increased to 86.70% for the PPC_{treated} sample. This result indicates the protein aggregate powder sample by combined ACP and pH-shifting treatment had good solubility. Despite the increased surface hydrophobicity, the strong surface charge (−31.50 mV) enabled good stability of the soluble aggregates in aqueous suspension. The increase in solubility of PPC_{treated} sample seems contradictory to the formation of large aggregates in TEM results. This might be attributed to the unfolded protein structures after combined pH-shifting and ACP treatment. The unfolding of protein might expose other active sites on the protein surface that could promote the affinity with water molecules, leading to increased solubility. Similarly, the increase in the number of large fractions accompanied by improved solubility was observed for peanut protein after 2 min-ACP treatment in a previous study (Ji et al., 2018). The author explained that water micelles would be coupled with protein molecules to make larger protein micelles (Ji et al., 2018). As shown in FIG. 17B, the apparent viscosity of the PPC_{untreated}, PPC_{pH-shifting}, PPC_{ACP}, and PPC_{treated} suspensions decreased with increasing shear rate, indicating a shear-thinning behavior (Gousse, Chanzy, Cerrada, & Fleury, 2004). When the shear rate increases, the asymmetric dispersed molecules tend to align along the direction of the shear plane, thereby reducing frictional resistance and leading to a decrease in viscosity. The viscosity of the PPC_{ACP} dispersion was higher than PPC_{untreated} and PPC_{pH-shifting}, and the viscosity of the PPC_{treated} was highest at all tested shear rates among these samples. This further suggests the connection of pea protein aggregates to form some level of crosslinking, in accordance with the TEM observation.

### Preparation of Pea Protein Gels

PPC_{untreated}, PPC_{pH-shifting}, and PPC_{treated} powders were dispersed in distilled water with the final protein content of 14 wt. %. Afterward, the protein suspensions were incubated in a water bath at 70, 80, and 90° C. for 10 and 20 min, respectively. Since PPC_{untreated} and PPC_{pH-shifting} only could not form gels or only form very weak gels, their protein content was increased to 18 wt. % and heated at 95° C. for 60 min to form self-standing gels for comparison. After gelation, the samples were immediately cooled in an ice-water bath for 10 min and stored in a fridge (4° C.) overnight before characterization. The prepared gel samples were labeled according to their preparation conditions, for example, T-70-10 and N-95-60 represent the gels prepared from PPC_{treated} at 70° C. for 10 min, and PPC_{untreated} at 95° C. for 60min, respectively.

### Characterizations of the PPC Gels

### Mechanical Properties and Textual Profile Analysis (TPA)

Mechanical properties and TPA of the prepared gels were evaluated by an Instron 5967 universal testing instrument (Instron Corp., MA, USA) equipped with a 50 N load cell. Cylindrical geometries of each sample (14 mm diameter and 8mm height) were compressed to 70% strain with a deformation rate of 1 mm/min at room temperature. The stress-strain curve was obtained to calculate the compressive strength and compressive strain, which are the maximum stress and strain at break or at 70%. For TPA analysis, the gel samples were compressed twice to 40% strain with 1 mm/min speed. Textural parameters including hardness, cohesiveness, springiness, and chewiness were calculated according to the method of Ferreira et al. (Ferreira, Calvinho, Cabrita, Schacht, & Gil, 2006) using OriginPro 8.5 software (Origin Lab, USA). Hardness was obtained at the peak force during the first compression cycle. Cohesiveness is the ratio Area 2/Area 1 in the force-time curve. Springiness was represented by Distance 2/Distance 1 in the force-time curve. The chewiness was calculated as hardness multiplied by cohesiveness and springiness.

As shown in FIG. 18, PPC_{untreated} could not form a gel at 14 wt. % of pea protein concentration, even heated to 90° C. for 20 min. The self-standing gels were only formed until reaching the protein concentration of 18 wt. %, heating temperature of 95° C. for 60 min. PPC_{pH-shifting} (very short pH-shifting time, only 5 min) suspensions of 18 wt. % did not form a gel even at 95° C. for 60 min. Short alkaline pH exposure did not lead to great significant changes in surface hydrophobicity, —SH groups, when compared to untreated samples. In addition, no aggregation was observed by TEM for the sample by 5 min pH-shifting. While for the PPC_{treated}, self-standing gels were formed already at a protein concentration of 14% when the temperature was increased to 70° C., and then the gels were strengthened with increase in temperature until 90° C. Further increasing temperature to 95° C. only increased the gel strength slightly. Therefore, we further studied the gel properties and gelling mechanism of PPC_{treated} suspension (14 wt. %) at various heating temperatures (70, 80, and 90° C.) and times (10 and 20 min). The gels prepared from PPC_{untreated} (18 wt. %) heated at 95° C. for 60 min were also characterized for comparison.

As shown in FIGS. 19A and B, the compressive strength and compressive strain were 1.90 kPa and 46.88%, respectively, for the gels prepared from 18 wt. % of PPC_{untreated} (95° C., 60 min). Comparable values were obtained for the gels formed from 14 wt. % of PPC_{treated} heated at 70° C. for 10 min, but lower than other PPC_{treated} gels. For example, the gels formed from PPC_{treated} under 80° C. for 20 min had a compressive strength of 3.70 kPa and a compressive strain of 58.02%. The compressive strength increased to 5.53 kPa when further raising the temperature to 90° C. Both the gel compressive strength and strain values were increased with the rise of the heating temperatures and times, resulting from more physical interactions and covalent bonds formation with increasing the temperatures and times (K.-Q. Wang et al., 2017). Liu et al. (Liu, Geng, Zhao, Chen, & Kong, 2015) modified soy protein isolate by combining acid pH-shifting (pH 1.5 for 5 h) and mild heating (60° C.) treatment, and the treated protein formed gels with the compressive strength of 2.86 kPa when heated at 90° C. for 30 min. This value was comparable to that PPC_{treated} gel formed at 70° C. for 20 min (2.81 kPa). In our previous study (S. Zhang et al., 2020), gels with lower compressive strength were obtained when ACP treatment alone was applied. For example, after ACP treatment the pea protein concentrates formed very weak gels (compressive strength: 0.53 kPa) when heated at 70° C. for 30 min. Whereas in this work, the compressive strength of PPC_{treated} gels formed at 70° C. for 20 min was increased more than 5 folds to 2.81 kPa. It is worthy of mentioning that the pH-shifting treatment would bring salt to the gel system, which might impact the protein gelation, but it was low (˜0.04% (w/w)) in this work, thus would not significantly change the gel properties (Shand, Ya, Pietrasik, & Wanasundara, 2007).

From the above results, gels were compressed to 40% of their height to study the textural profiles, and the gels prepared from PPC_{treated} and PPC_{untreated} were focused. The TPA test simulated oral mastication performed on the food matrix and the results are present in Table 2. Hardness is the resistance from solid material to other permanent deformation when compression is applied. Chewiness indicates the energy required to chew solid food into swallow-able food. Springiness reflects the ability to spring back to the original shape after removing the applied force. Cohesiveness signifies the internal binding force required for food formation. TPA values of the gels prepared from PPC_{treated} of 14 wt. % at 70° C. for 20 min or at 80-90° C. for 10 min were comparable or even higher than the gels from PPC_{untreated} (18 wt. %) heated at 95° C. for 60 min. For example, the PPC_{treated} gels heated at 80° C. for only 10 min and 70° C. for 20 min showed comparable harness and chewiness values to the PPC_{untreated} gels (60 min heating at 95° C.), while significantly higher cohesiveness and springiness values. High temperature and long heating time are required for pea protein (without treatment) to form gels because the pea legumin and vicilin proteins have high denaturation temperature (legumin: ˜92° C., vicilin: ˜83° C.) (Liang & Tang, 2013) due to their compact molecular structure. However, such intensive heating can be difficult to achieve by some industrial food processing. For example, the internal temperature of vegan burgers reaches only ˜75° C. during the cooking process (Services, 2017). The significantly reduced gelling temperature to 70-80° C. will allow the PPC-based gelling ingredient to be applied in many plant protein-based food products. The significantly reduced heating time from 60 min to 10-20 min also makes the gelling process more energy efficient.

TABLE 2
Textural profile analysis (TPA) of pea protein concentrate (PPC) gels
with (T) or without (N) atmospheric cold plasma (ACP) treatment in
combination with pH-shifting compressed twice to 40% strain.

| Samples | Hardness (N) | Cohesiveness | Springiness | Chewiness (N) |
|---|---|---|---|---|
| T-70-10 | 0.140 ± 0.014^{a} | 0.745 ± 0.007^{ab} | 0.815 ± 0.007^{b} | 0.085 ± 0.009^{a} |
| T-70-20 | 0.180 ± 0.014^{ab} | 0.750 ± 0.014^{b} | 0.790 ± 0.014^{b} | 0.104 ± 0.008^{ab} |
| T-80-10 | 0.210 ± 0.014^{b} | 0.745 ± 0.021^{ab} | 0.805 ± 0.021^{b} | 0.126 ± 0.008^{b} |
| T-80-20 | 0.215 ± 0.007^{b} | 0.750 ± 0.014^{b} | 0.790 ± 0.014^{b} | 0.127 ± 0.009^{b} |
| T-90-10 | 0.215 ± 0.035^{b} | 0.725 ± 0.064^{ab} | 0.815 ± 0.035^{b} | 0.126 ± 0.004^{b} |
| T-90-20 | 0.215 ± 0.007^{b} | 0.755 ± 0.021^{b} | 0.810 ± 0.028^{b} | 0.132 ± 0.013^{b} |
| N-95-60 | 0.250 ± 0.042^{b} | 0.670 ± 0.042^{a} | 0.730 ± 0.014^{a} | 0.122 ± 0.015^{b} |

Different lower-case letters within a column denote significant differences (p < 0.05), as determined by One-way analysis of variance (ANOVA) followed by a post-hoc test (Fisher's Least Significant Difference (LSD)).

### Rheological Properties

Rheological behaviors of gels were studied by a TA Discovery HR-3 rheometer (TA Instruments, DE, USA) equipped with a parallel-plate geometry (diameter of 40 mm) and the gap was set at 1 mm. The gel sample was placed on the rheometer plate for the frequency sweep test. The storage modulus (G′) and loss modulus (G″) were recorded at oscillation frequency from 0.1 to 100 rad/s under a constant strain of 1%. For dynamic viscoelastic measurements, the temperature ramp test was conducted to study the relationship between viscoelastic modulus and temperature. Approximately 1.5 mL of PPC_{untreated} and PPC_{treated} suspensions (14 wt. %) were heated from 25 to respective 70, 80, and 90° C. at a rate of 2° C./min for 10 min, cooled down to 4° C. at 4° C./min and kept for 20 min under the frequency of 1 Hz and the strain value at 1%, which was within a predetermined linear viscoelastic region. Silicone oil and a solvent trap cover were used to prevent evaporation and keep a thermally stable environment during the measurements. G′ and G″ were obtained continually.

Rheological properties of PPC suspensions were studied with frequency sweep measurement in the limitation of the linear viscoelastic region (LVR) (FIG. 19C). As shown in FIG. 19C, G′ was lower than G″ within all frequencies tested for PPC_{untreated} suspension, exhibiting liquid behavior in the sample. Regarding the PPC_{treated} suspension, G′ was lower than G″ at low frequencies, and both moduli increased with the frequency increasing, indicating a highly elastic solution due to ordered protein chain segments (T. Zhang, Jiang, & Wang, 2007). This result was in accordance with the TEM observation that structured fluids were formed by connected pea protein aggregates by combined ACP and pH-shifting treatment. Rheological properties of PPC gels were also evaluated. PPC_{untreated} and PPC_{treated} gel samples behaved as gel-like materials where G′ were prevailing over G″ with all tested oscillation frequencies. Bonds between protein molecules may be formed and broken during the frequency sweep test, which affects rheological properties (G′ and G″) due to the structural changes (Tunick, 2011). The G′ value of PPC_{treated} gels was little dependent on frequency, whereas both G′ and G″ of PPC_{untreated} gel were frequency-dependent and got closer to each other at high frequency. This result further confirms that significantly strengthened gels were obtained from pea protein by combined ACP and pH-shifting treatment. Brito-Oliveira et al. (Brito-Oliveira, Bispo, Moraes, Campanella, & Pinho, 2018) pre-heated soy protein isolate with 15% (w/v) at 80° C. for 30 min, followed by adding CaCl2 solution (10 mM of final concentration) to obtain a self-supported gel. The G′ values of this soy protein gel with all frequencies were around 10² Pa, while the G′ values of all PPC_{treated} gels were higher than 10 2 Pa, even for the gel heated at 70° C. for 10 min.

### Water Holding Capacity and Gel Microstructure

Water holding capacity (WHC) of the gels was measured by the method of Yang et al. (Yang, Wang, & Chen, 2017) with modifications. About 0.5 g gel sample was transferred into a Vivaspin 20 centrifugal filter unit (GE Healthcare Bio-Sciences AB, Uppsala, Sweden), then centrifuged at 4,000×g for 30 min. The weight of the system of tubes and gel samples before and after centrifugation were recorded. WHC was calculated by Equation (3).

$$WHC = \frac{{W}_{1} - {W}_{2}}{{W}_{1}} \times 100 \% \qquad ( 3 )$$

where W₁ is the weight of total water in the gel before centrifugation (g), W₂ is the weight of water released in the gel by centrifugation (g).

Water holding capacity (WHC) is the ability to interact with water in a gel and is an important quality attribute of food products. For example, the water holding capacity of the meat and meat analogue products is associated with juiciness and texture (Y. Jiang et al., 2021). Excellent water holding capacity was observed for both PPC_{untreated} and PPC_{treated} gels with the WHC value of 93.36-98.18% (FIG. 18D).

The microstructures of PPC_{untreated} and PPC_{treated} gels were observed by scanning electron microscopy (EVO M10 scanning electron microscope (SEM) (Carl Zeiss AG, Oberkochen, Germany) at an acceleration voltage of 20 kV and magnifications of 1500 and 5000. Each gel sample was quickly frozen in liquid nitrogen till it solidified well before freeze-drying. The cross-sections of the gel samples were fixed on a metal platform with conductive tape and sputter-coated with gold for 90 s in a vacuum coating unit before observation.

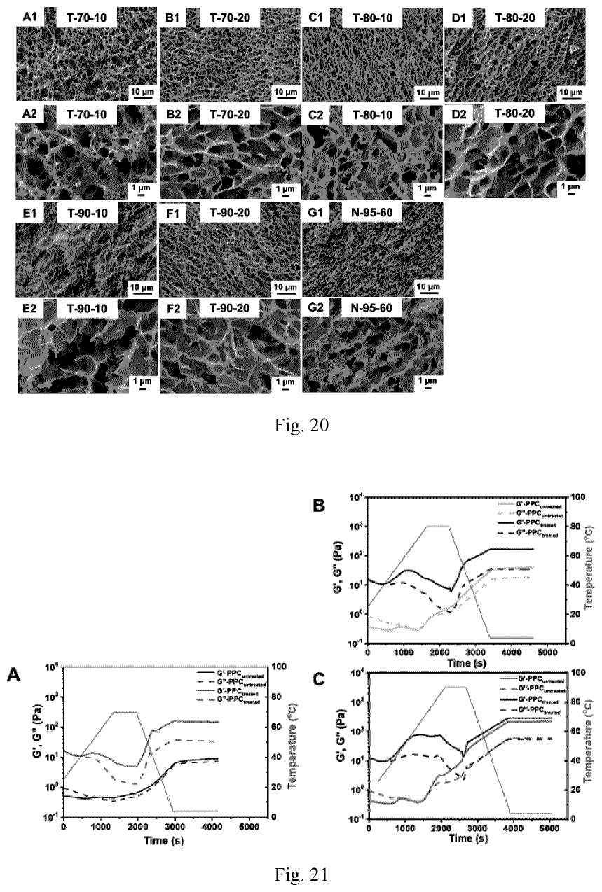

A uniform and dense gel network with neatly arranged pores was obtained from PPC_{treated} even at 70° C. for 20 min (FIG. 20B). The PPC_{treated} gel structures became more compact with increasing heating temperature and time, explaining the trend observed for the gel compressive strength. In addition, thicker walls around the pores were observed in the PPC_{treated} gels formed at a higher temperature and longer time. For comparison, the gels prepared from PPC_{untreated} (18 wt. %) by heating at 95° C. for 60 min showed dense crosslinked network, but coarse wall structure (FIG. 5G). The relative speed of protein unfolding and aggregation play an important role in deciding the microstructure of heat-induced protein gels. If the speed of aggregation is faster than that of protein unfolding, gel microstructure would be denser and uniform and vice versa (F. Lefèvre, Fauconneau, Ouali, & Culioli, 1998). PPC_{untreated} requires heating at 95° C. for 60 min to form self-standing gels, however, high temperature accelerated protein aggregation after unfolding, thus the gel networks were developed from coarser protein aggregates. For PPC_{treated}, lower heating temperature (70-90° C.) might favor a balanced unfolding and aggregation speed. In addition, the soluble aggregates in PPC_{treated} suspension facilitate aggregate growth in a more ordered structure, leading to a more uniform and highly crosslinked network, subsequently gels with improved strength.

### Gel Formation Mechanism

Molecular forces involved in the PPC gel formation were analyzed according to the method of Nieto-Nieto et al. (Nieto-Nieto, Wang, Ozimek, & Chen, 2015). Gel samples with cylindrical geometries (1.0 cm height and 1.4 cm diameter) were immersed into water (as a control), 0.6 M 2-mercaptoethanol (2-ME), 6 M urea, and 3% (w/v) sodium dodecyl sulfate (SDS) solution for 48 h at room temperature respectively. G′ and G″ of soaked gels were recorded with a frequency sweep analysis.

FIGS. 21A, B, and C illustrate the rheological properties of PPC_{untreated} and PPC_{treated} (14 wt. %) heated to 70, 80, and 90° C. at 2° C./min, then held for 10 min, followed by cooling to 4° C. for 20 min, respectively. The curve of G′ and G″ for PPC_{untreated} heated to 70° C. were closer to each other (FIG. 21A), indicating that heating temperature of 70° C. was not sufficient to form a strong gel network with solid-like behavior. The PPC_{untreated} heated to 80 and 90° C. exhibited a similar trend of moduli change (FIGS. 21B and C), but different from that of PPC_{untreated} at 70° C. (FIG. 21A). As the temperature increased, G′ began to rise higher than G″ at about 80° C., which indicates that the network began to form. Both moduli continued to rise during further heating and cooling period, indicating further development of protein interactions (J. Chen & Dickinson, 2000). For PPC_{treated} suspensions, the G′ and G″ showed similar values at the beginning, followed by a slight decrease with increasing heating temperature until 40° C., due to the disruption of interactions in the aggregates by initial heating (K.-Q. Wang et al., 2017). Higher G′ than G″ was observed for PPC_{treated} at over 40° C., indicating that the solid network began to form at low temperature from the aggregate suspension. As the temperature increased to 70, 80, and 90° C., the G′ values increased due to the enhanced interaction between the aggregates in the gel network. This result suggests that soluble aggregates could grow into a solid network at temperatures as low as 40° C. for PPC_{treated}. This avoided rapid protein unfolding and aggregation when heated at high temperature (95° C.), and the formation of coarse gel networks. Finally, a remarkable escalation of G′ for PPC_{treated} occurred when the temperature was reduced to 4° C., indicating further development of the gel network by cooling, possibly due to the increased hydrogen bonding at low temperature (Zhao, Yu, Hemar, Chen, & Cui, 2020).

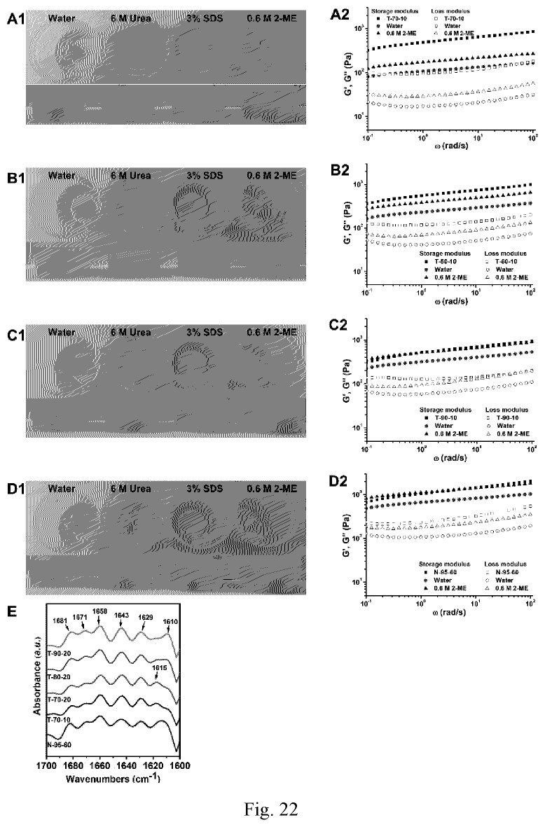

Gels were then immersed into different dissociation agents for 48 h including 6 M of urea, 3% of sodium dodecyl sulfate (SDS), and 0.6 M of 2-mercaptoethanol (2-ME) to break the hydrogen bonds, hydrophobic interactions, and disulfide bonds, respectively and the photographs are presented in FIGS. 22A1, B1, C1, and D1. The gels soaked into urea and SDS agents were destroyed, suggesting that both hydrogen bonds and hydrophobic interactions played important roles in the formation of gels from both PPC_{untreated} and PPC_{treated}. Since the gels soaked into water and 2-ME still maintained the original shape, a frequency sweep test was conducted to study the change of the gel rheological properties. As shown in FIGS. 7A2, B2, C2, and D2, the G′ of tested gels soaked into 2-ME solvent was higher than that of gels soaked in water, probably resulting in the replacement of water in the gel by 2-ME. This result suggests that although disulfide bonds were involved in pea protein aggregation by combined ACP treatment and pH-shifting method, they did not significantly contribute to gel network stabilization, possibly due to the fact that the cysteine content in pea protein is relatively low and the —SH groups were almost all engaged in the aggregate formation (Pownall, Udenigwe, & Aluko, 2010).

The FTIR spectra (FIG. 22E) were recorded for the gels prepared from PPC_{untreated} by heating at 95° C. for 60 min. The absorption associate with protein aggregation at 1619 cm⁻¹ and 1681 cm⁻appeared in the spectra of the gels formed from PPC_{untreated}. Many secondary structure components of PPC_{untreated} were still maintained after gel formation. This suggests that full unfolding of pea protein did not occur even after heating at 95° C. for 60 min probably due to the compact structure of the untreated pea protein. This might be another reason to explain the weak gelling capacity of pea protein.

Similar FTIR profiles were observed for the gels from PPC_{treated} suspensions. The co-existence of the absorptions at 1619 and 1681cm⁻¹ and the protein α-helix and β-sheet at 1658 cm⁻¹ and 1629 cm⁻¹ in the gel spectra suggests that the formed aggregates in PPC_{untreated} suspension did not undergo extensive unfolding by heating during the gel formation. Instead, the gel networks were built from the association of the aggregate. Since the aggregates were highly soluble, while possessing higher surface hydrophobicity, they could serve as active building blocks to gradually grow into a more ordered gel network with temperature increasing from 40° C. to 90° C. The gel structure was further enhanced during the cooling process.

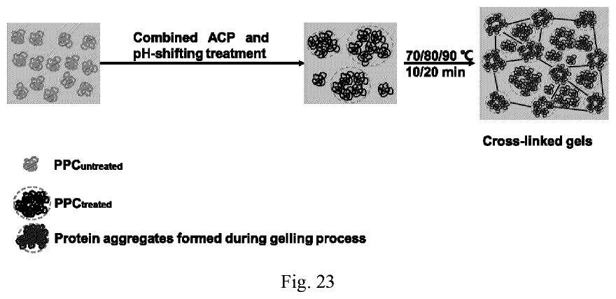

Based on the above results, the PPC_{treated} gelation mechanism is proposed in FIG. 23. Combined ACP and pH-shifting treatment partially unfolded pea protein by altering the tertiary structure. Then the active species produced by ACP facilitated the reaction between partially unfolded protein molecules, resulting in the formation of protein aggregates. Immediately after the treatment, the pH was adjusted back to neutral, followed by spray-drying to prepare PPC_{treated} powders. In spite of the increased surface hydrophobicity, the aggregates showed good solubility when rehydrated due to the substantial surface charge. These properties allowed them to serve as active building blocks to associate into more ordered 3D gel networks via hydrophobic interactions by heating even at low temperatures with a short period of time, such as 70° C. for 10 min. The hydrogen bonding also significantly contributed to the gel network stabilization, especially during the cooling stage.

### Statistical Analysis

All the gel preparations were repeated three times. All results are expressed as the mean±standard deviation (SD) from three independent batches. Statistical analysis was examined by a One-way analysis of variance (ANOVA) followed by a post-hoc test (Fisher's Least Significant Difference (LSD)) using SPSS Statistics 21.0 (IBM Corp, USA) with significance defined at p<0.05.

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated. To the extent that the following description is of a specific embodiment or a particular use of the invention, it is intended to be illustrative only, and not limiting of the claimed invention.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to combine, affect or connect such aspect, feature, structure, or characteristic with other embodiments, whether or not such connection or combination is explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all ranges described herein, and all language such as “up to”, “at least”, “greater than”, “less than”, “more than”, “or more”, and the like, include the number(s) recited and such terms refer to ranges that can be subsequently broken down into sub-ranges as discussed above.

References—the following references are provided to indicate the level of skill of one skilled in the art, and are incorporated herein by reference in their entirety. Any inconsistency between any reference and the disclosure herein may be considered to be alternative information.
Abaee, A., Mohammadian, M., & Jafari, S. M. (2017). Whey and soy protein-based hydrogels and nano-hydrogels as bioactive delivery systems. Trends in Food Science & Technology, 70, 69-81. https://doi.org/10.1016/j.tifs.2017.10.011.
Bahrami, N., Bayliss, D., Chope, G., Penson, S., Perehinec, T., & Fisk, I. D. (2016). Cold plasma: A new technology to modify wheat flour functionality. Food Chemistry, 202, 247-253. https://doi.org/10.1016/j.foodchem.2016.01.113.
Bard, D. Update on Canada's Pulse Crops. (2019). https://pulsepod.globalpulses.com/pod-feed/post/update-on-canada-pulse-crops.
Ben-Harb, S., Panouillé, M., Huc-Mathis, D., Moulin, G., Saint-Eve, A., Irlinger, F., Bonnarme, P., Michon, C., & Souchon, I. (2018). The rheological and microstructural properties of pea, milk, mixed pea/milk gels and gelled emulsions designed by thermal, acid, and enzyme treatments. Food Hydrocolloids, 77, 75-84. https://doi.org/10.1016/j.foodhyd.2017.09.022.
Bourke, P., Ziuzina, D., Boehm, D., Cullen, P. J., & Keener, K. (2018). The potential of cold plasma for safe and sustainable food production. Trends in Biotechnology, 36(6), 615-626.
Bradford, M. M. (1976). A rapid and sensitive method for the quantitation of microgram quantities of protein utilizing the principle of protein-dye binding. Analytical Biochemistry, 72 (1-2), 248-254. Braga, A. L. M., Azevedo, A., Marques, M. J., Menossi, M., & Cunha, R. L. (2006). Interactions between soy protein isolate and xanthan in heat-induced gels: The effect of salt addition. Food Hydrocolloids, 20(8), 1178-1189. https://doi.org/10.1016/j.foodhyd.2006.01.003.
Brito-Oliveira, T. C., Bispo, M., Moraes, I. C., Campanella, O. H., & Pinho, S. C. (2018). Cold-set gelation of commercial soy protein isolate: Effects of the incorporation of locust bean gum and solid lipid microparticles on the properties of gels. Food Biophysics, 13(3), 226-239. https://doi.org/10.1007/s11483-018-9529-4.
Bryant, C. M., & McClements, D. J. (1998). Molecular basis of protein functionality with special consideration of cold-set gels derived from heat-denatured whey. Trends in Food Science & Technology, 9(4), 143-151. https://doi.org/10.1016/S0924-2244(98)00031-4.
Buβler, S., Steins, V., Ehlbeck, J. and Schlüter, O. (2015). Impact of thermal treatment versus cold atmospheric plasma processing on the techno-functional protein properties from Pisum sativum ‘Salamanca’. Journal of Food Engineering, 167, 166-174. https://doi.org/10.1016/j.jfoodeng.2015.05.036.
Byler, D. M., & Susi, H. (1986). Examination of the secondary structure of proteins by deconvolved FTIR spectra. Biopolymers: Original Research on Biomolecules, 25(3), 469-487. https://doi-org/10.1002/bip.360250307.
Cao, Y, & Xiong, Y. L. (2015). Chlorogenic acid-mediated gel formation of oxidatively stressed myofibrillar protein. Food Chemistry, 180, 235-243. https://doi.org/10.1016/j.foodchem.2015.02.036.
Chalamaiah, M., Esparza, Y., Temelli, F., & Wu, J. (2017). Physicochemical and functional properties of livetins fraction from hen egg yolk. Food Bioscience, 18, 38-45. https://doi.org/10.1016/j.fbio.2017.04.002.
Chen, J., & Dickinson, E. (2000). On the temperature reversibility of the viscoelasticity of acid-induced sodium caseinate emulsion gels. International Dairy Journal, 10(8), 541-549.
Chen, W., Wang, W., Ma, X., Lv, R., Watharkar, R. B., Ding, T., Ye, X., & Liu, D. (2019). Effect of pH-shifting treatment on structural and functional properties of whey protein isolate and its interaction with (-)-epigallocatechin-3-gallate. Food Chemistry, 274, 234-241.
Chihi, M.-L., Mession, J.-l., Sok, N., & Saurel, R. m. (2016). Heat-induced soluble protein aggregates from mixed pea globulins and β-lactoglobulin. Journal of Agricultural and Food Chemistry, 64(13), 2780-2791.
Chizoba Ekezie, F.-G, Cheng, J.-H., & Sun, D.-W. (2018). Effects of Mild Oxidative and Structural Modifications Induced by Argon Plasma on Physicochemical Properties of Actomyosin from King Prawn (Litopenaeus vannamei). Journal of Agricultural and Food Chemistry, 66(50), 13285-13294. https://doi.org/10.1021/acs.jafc.8b05178.
Clark, A. H., Kavanagh, G. M., & Ross-Murphy, S. B. (2001). Globular protein gelation—theory and experiment. Food Hydrocolloids, 15(4-6), 383-400. https://doi.org/10.1016/S0268-005X(01)00042-X.
Croy, R., Gatehouse, J. A., Tyler, M., & Boulter, D. (1980). The purification and characterization of a third storage protein (convicilin) from the seeds of pea (Pisum sativum L.). Biochemical Journal, 191(2), 509-516. https://doi.org/10.1042/bj1910509.
Damodaran, S. (2008). Amino acids, peptides and proteins. In S. Damodaran, K.L. Parkin, & O.R. Fennema (Eds.), Food Chemistry, (pp. 242-245). CRC Press.
de Oliveira, A. P. H., Omura, M. H., Barbosa, É. d. A. A., Bressan, G. C., Vieira, É. N. R., dos Reis Coimbra, J. S., & de Oliveira, E. B. (2020). Combined adjustment of pH and ultrasound treatments modify techno-functionalities of pea protein concentrates. Colloids and Surfaces A: Physicochemical and Engineering Aspects, 603, 125156.
Deng, S., Ruan, R., Mok, C. K., Huang, G, Lin, X., & Chen, P. (2007). Inactivation of Escherichia coli on almonds using nonthermal plasma. Journal of Food Science, 72(2), M62-M66. https://doi.org/10.1111/j.1750-3841.2007.00275.x.
DHHS. Food safety when cooking. (2017). https://www.betterhealth.vic.gov.au/health/healthyliving/food-safety-when-cooking.
Dong, S., Gao, A., Xu, H., & Chen, Y. (2017). Effects of dielectric barrier discharges (DBD) cold plasma treatment on physicochemical and structural properties of zein powders. Food and Bioprocess Technology, 10(3), 434-444. https://doi.org/10.1007/s11947-017-2015-z.
Dong, S., Guo, P., Chen, Y., Chen, G-y., Ji, H., Ran, Y., Li, S.-h., & Chen, Y. (2018). Surface modification via atmospheric cold plasma (ACP): improved functional properties and characterization of zein film. Industrial Crops and Products, 115, 124-133. https://doi.org/10.1016/j.indcrop.2018.01.080.
Dong, S., Wang, J.-m., Cheng, L.-m., Lu, Y.-l., Li, S.-h., & Chen, Y. (2017). Behavior of zein in aqueous ethanol under atmospheric pressure cold plasma treatment. Journal of Agricultural and Food Chemistry, 65(34), 7352-7360. https://doi.org/10.1021/acs.jafc.7b02205.
Ellepola, S. W., Choi, S. M., & Ma, C. Y. (2005). Conformational study of globulin from rice (Oryza sativa) seeds by Fourier-transform infrared spectroscopy. International Journal of Biological Macromolecules, 37(1-2), 12-20.
Feizollahi, E., Arshad, M., Yadav, B., Ullah, A., & Roopesh, M. (2020). Degradation of deoxynivalenol by atmospheric-pressure cold plasma and sequential treatments with heat and UV light. Food Engineering Reviews, 1-10.
Feizollahi, E., Misra, N., & Roopesh, M. (2020). Factors influencing the antimicrobial efficacy of Dielectric Barrier Discharge (DBD) Atmospheric Cold Plasma (ACP) in food processing applications. Critical Reviews in Food Science and Nutrition, 1-24. https://doi.org/10.1080/10408398.2020.1743967.
Ferreira, P., Calvinho, P., Cabrita, A. S., Schacht, E., & Gil, M. H. (2006). Synthesis and characterization of new methacrylate based hydrogels. Revista Brasileira de Ciências Farmacêuticas, 42(3), 419-427. https://doi.org/10.1590/S1516-93322006000300011.
Figueroa-González, J. J., Lobato-Calleros, C., Vernon-Carter, E. J., Aguirre-Mandujano, E., Alvarez-Ramirez, J., & Martinez-Velasco, A. (2022). Modifying the structure, physicochemical properties, and foaming ability of amaranth protein by dual pH-shifting and ultrasound treatments. LWT—Food Science and Technology, 153, 112561.
Forte, A. E., D'amico, F., Charalambides, M. N., Dini, D., & Williams, J. G. (2015). Modelling and experimental characterisation of the rate dependent fracture properties of gelatine gels. Food Hydrocolloids, 46, 180-190. https://doi.org/10.1016/j.foodhyd.2014.12.028.
Fujihara, S., Kasuga, A., & Aoyagi, Y. (2001). Nitrogen-to-protein conversion factors for common vegetables in Japan. Journal of Food Science, 66(3), 412-415. https://doi.org/10.1111/j.1365-2621.2001.tb16119.x.
Gousse, C., Chanzy, H., Cerrada, M., & Fleury, E. (2004). Surface silylation of cellulose microfibrils: preparation and rheological properties. Polymer; 45(5), 1569-1575.
Gueguen, J., & Cerletti, P. (1994). Proteins of some legume seeds: soybean, pea, fababean and lupin. In B.J.F. Hudson (Ed.), New and Developing Sources of Food Proteins (pp. 145-183). Springer Science & Business Media Dordrecht.
Habeeb, A. S. A. (1966). Determination of free amino groups in proteins by trinitrobenzenesulfonic acid. Analytical Biochemistry, 14(3), 328-336. https://doi.org/10.1016/0003-2697(66)90275-2.
Han, Y., Cheng, J.-H., & Sun, D.-W. (2019). Activities and conformation changes of food enzymes induced by cold plasma: A review. Critical Reviews in Food Science and Nutrition, 59(5), 794-811.
Hartman, H. T., Kofranek, A. M., Rubatzky, V. E., & Flocker, W. J. (1988). Plant Science. Growth, Development, and Utilization of Cultivated Plants: Prentice-Hall.
Haskard, C. A., & Li-Chan, E. C. (1998). Hydrophobicity of bovine serum albumin and ovalbumin determined using uncharged (PRODAN) and anionic (ANS-) fluorescent probes. Journal of Agricultural and Food Chemistry, 46 (7), 2671-2677.
He, J. S., Azuma, N., & Yang, H. (2010). Effects of pH and ionic strength on the rheology and microstructure of a pressure-induced whey protein gel. International Dairy Journal, 20(2), 89-95. https://doi.org/10.1016/j.idairyj.2009.08.006.
He, S., Zhao, J., Cao, X., Ye, Y., Wu, Z., Yue, J., Yang, L., Jin, R., & Sun, H. (2020). Low pH-shifting treatment would improve functional properties of black turtle bean (Phaseolus vulgaris L.) protein isolate with immunoreactivity reduction. Food Chemistry, 330, 127217.
Hermansson, A. M. (1978). Physico-chemical aspects of soy proteins structure formation. Journal of Texture Studies, 9(1-2), 33-58. https://doi-org/10.1111/j.1745-4603.1978.tb01293.x.
Holt, N. W., & Sosulski, F. W. (1979). Amino acid composition and protein quality of field peas. Canadian Journal of Plant Science, 59(3), 653-660. https://doi-org/10.4141/cjps79-103.
Ji, H., Dong, S., Han, F., Li, Y., Chen, G., Li, L., & Chen, Y. (2018). Effects of dielectric barrier discharge (DBD) cold plasma treatment on physicochemical and functional properties of peanut protein. Food and Bioprocess Technology, 11(2), 344-354. https://doi.org/10.1007/s11947-017-2015-z.
Ji, H., Han, F., Peng, S., Yu, J., Li, L., Liu, Y., Chen, Y, Li, S., & Chen, Y. (2019). Behavioral Solubilization of Peanut Protein Isolate by Atmospheric Pressure Cold Plasma (ACP) Treatment. Food and Bioprocess Technology, 12(12), 2018-2027. https://doi.org/10.1007/s11947-019-02357-0.
Jiang, J., Chen, J., & Xiong, Y. L. (2009). Structural and emulsifying properties of soy protein isolate subjected to acid and alkaline pH-shifting processes. Journal ofAgricultural and Food Chemistry, 57(16), 7576-7583. https://doi.org/10.1021/jf901585n.
Jiang, J., Xiong, Y. L., & Chen, J. (2010). pH shifting alters solubility characteristics and thermal stability of soy protein isolate and its globulin fractions in different pH, salt concentration, and temperature conditions. Journal of Agricultural and Food Chemistry, 58(13), 8035-8042. https://doi.org/10.1021/jf101045b.
Jiang, J., Zhu, B., Liu, Y., & Xiong, Y. L. (2014). Correction to interfacial structural role of pH-shifting processed pea protein in the oxidative stability of oil/water emulsions. Journal of Agricultural and Food Chemistry, 62(18), 4225. https://doi.org/10.1021/jf501898m.
Jiang, J., Zhu, B., Liu, Y, & Xiong, Y. L. (2014). Interfacial structural role of pH-shifting processed pea protein in the oxidative stability of oil/water emulsions. Journal of Agricultural and Food Chemistry, 62(7), 1683-1691.
Jiang, S., Ding, J., Andrade, J., Rababah, T. M., Almajwal, A., Abulmeaty, M. M., & Feng, H. (2017). Modifying the physicochemical properties of pea protein by pH-shifting and ultrasound combined treatments. Ultrasonics Sonochemistry, 38, 835-842. https://doi.org/10.1016/j.ultsonch.2017.03.046.
Jiang, Y., Li, D., Tu, J., Zhong, Y., Zhang, D., Wang, Z., & Tao, X. (2021). Mechanisms of change in gel water-holding capacity of myofibrillar proteins affected by lipid oxidation: The role of protein unfolding and cross-linking. Food Chemistry, 344, 128587.
Kato, A., & Nakai, S. (1980). Hydrophobicity determined by a fluorescence probe method and its correlation with surface properties of proteins. Biochimica et Biophysica Acta (BBA)-Protein Structure, 624(1), 13-20. https://doi.org/10.1016/0005-2795(80)90220-2.
Khemakhem, M., Attia, H., & Ayadi, M. A. (2019). The effect of pH, sucrose, salt and hydrocolloid gums on the gelling properties and water holding capacity of egg white gel. Food Hydrocolloids, 87, 11-19. https://doi.org/10.1016/j.foodhyd.2018.07.041.
Kim, J. E., Lee, D.-U., & Min, S. C. (2014). Microbial decontamination of red pepper powder by cold plasma. Food Microbiology, 38, 128-136. https://doi.org/10.1016/j.fm.2013.08.019.
Klost, M., Brzeski, C., & Drusch, S. (2020). Effect of protein aggregation on rheological properties of pea protein gels. Food Hydrocolloids, 106036.
Kocher, P. N., & Foegeding, E. A. (1993). Microcentrifuge-based method for measuring water-holding of protein gels. Journal of Food Science, 58(5), 1040-1046. https://doi.org/10.1111/j.1365-2621.1993.tb06107.x.
Kuwajima, K. (1989). The molten globule state as a clue for understanding the folding and cooperativity of globular-protein structure. Proteins: Structure, Function, and Bioinformatics, 6(2), 87-103.
Lefèvre, F., Fauconneau, B., Ouali, A., & Culioli, J. (1998). Thermal Gelation of Brown Trout Myofibrils: Effect of Muscle Type, Heating Rate and Protein Concentration. Journal of Food Science, 63(2), 299-304.
Lefèvre, T., & Subirade, M. (1999). Structural and interaction properties of β-Lactoglobulin as studied by FTIR spectroscopy. International Journal of Food Science & Technology, 34(5-6), 419-428.
Lefèvre, T., & Subirade, M. (2000). Molecular differences in the formation and structure of fine-stranded and particulate β-lactoglobulin gels. Biopolymers: Original Research on Biomolecules, 54(7), 578-586. https://doi.org/10.1002/1097-0282(200012)54:7<578::AID-BIP100>3.0.CO;2-2.
Li, J., Wu, M., Wang, Y., Li, K., Du, J., & Bai, Y. (2020). Effect of pH-shifting treatment on structural and heat induced gel properties of peanut protein isolate. Food Chemistry, 325, 126921.
Liang, H.-N., & Tang, C.-H. (2013). pH-dependent emulsifying properties of pea [Pisum sativum (L.)] proteins. Food Hydrocolloids, 33(2), 309-319. https://doi.org/10.1016/j.foodhyd.2013.04.005.
Liu, Q., Geng, R., Zhao, J., Chen, Q., & Kong, B. (2015). Structural and gel textural properties of soy protein isolate when subjected to extreme acid pH-shifting and mild heating processes. Journal of Agricultural and Food Chemistry, 63(19), 4853-4861.
Ma, C. Y., Rout, M. K., Chan, W. M., & Phillips, D. L. (2000). Raman spectroscopic study of oat globulin conformation. Journal of Agricultural and Food Chemistry, 48(5), 1542-1547. https://doi.org/10.1021/jf991222n.
Maltais, A., Remondetto, G. E., Gonzalez, R., & Subirade, M. (2005). Formation of soy protein isolate cold-set gels: protein and salt effects. Journal of Food Science, 70(1), C67-C73. https://doi.org/10.1111/j.1365-2621.200511)09023.x.
Mandal, R., Singh, A., & Singh, A. P. (2018). Recent developments in cold plasma decontamination technology in the food industry. Trends in Food Science & Technology, 80, 93-103. https://doi.org/10.1016/j.tifs.2018.07.014.
Marfil, P. H., Anhê, A. C., & Telis, V. R. (2012). Texture and microstructure of gelatin/corn starch-based gummy confections. Food Biophysics, 7(3), 236-243. https://doi.org/10.1007/s11483-012-9262-3.
Martinez-López, A. L., Carvajal-Millan, E., Micard, V., Rascón-Chu, A., Brown-Bojorquez, F., Sotelo-Cruz, N., López-Franco, Y. L., & Lizardi-Mendoza, J. (2016). In vitro degradation of covalently cross-linked arabinoxylan hydrogels by bifidobacteria. Carbohydrate Polymers, 144, 76-82. https://doi.org/10.1016/j.carbpol.2016.02.031.
Mehr, H. M., & Koocheki, A. (2020). Effect of atmospheric cold plasma on structure, interfacial and emulsifying properties of Grass pea (Lathyrus sativus L.) protein isolate. Food Hydrocolloids, 106, 105899.
Mehr, H. M., & Koocheki, A. (2021). Physicochemical properties of Grass pea (Lathyrus sativus L.) protein nanoparticles fabricated by cold atmospheric-pressure plasma. Food Hydrocolloids, 112, 106328.
Miao, W., Nyaisaba, B. M., Koddy, J. K., Chen, M., Hatab, S., & Deng, S. (2020). Effect of cold atmospheric plasma on the physicochemical and functional properties of myofibrillar protein from Alaska pollock (Theragra chalcogramma). International Journal of Food Science & Technology, 55(2), 517-525. https://doi.org/10.1111/ijfs.14295.
Miriani, M., Iametti, S., Bonomi, F., & Corredig, M. (2012). Structural changes of soy proteins at the oil-water interface studied by fluorescence spectroscopy. Colloids and Surfaces B: Biointerfaces, 93, 41-48.
Misra, N., Kaur, S., Tiwari, B. K., Kaur, A., Singh, N., & Cullen, P. (2015). Atmospheric pressure cold plasma (ACP) treatment of wheat flour. Food Hydrocolloids, 44, 115-121.
Moreno, H. M., Dominguez-Timón, F., Diaz, M. T., Pedrosa, M. M., Borderias, A. J., & Tovar, C. A. (2020). Evaluation of gels made with different commercial pea protein isolate: Rheological, structural and functional properties. Food Hydrocolloids, 99, 105375. https://doi.org/10.1016/j.foodhyd.2019.105375.
Munialo, C. D., van der Linden, E., & de Jongh, H. H. (2014). The ability to store energy in pea protein gels is set by network dimensions smaller than 50 nm. Food Research International, 64, 482-491. https://doi.org/10.1016/j.foodres.2014.07.038.
Nieto-Nieto, T. V., Wang, Y., Ozimek, L., & Chen, L. (2016). Improved thermal gelation of oat protein with the formation of controlled phase-separated networks using dextrin and carrageenan polysaccharides. Food Research International, 82, 95-103. https://doi.org/10.1016/j.foodres.2016.01.027.
Nieto-Nieto, T. V., Wang, Y. X., Ozimek, L., & Chen, L. (2015). Inulin at low concentrations significantly improves the gelling properties of oat protein-A molecular mechanism study. Food Hydrocolloids, 50, 116-127. https://doi.org/10.1016/j.foodhyd.2015.03.031.
O'Kane, F. E., Happe, R. P., Vereijken, J. M., Gruppen, H., & van Boekel, M. A. (2004). Heat-induced gelation of pea legumin: comparison with soybean glycinin. Journal of Agricultural and Food Chemistry, 52(16), 5071-5078. https://doi.org/10.1021/jf035215h.
O'Kane, F. E., Vereijken, J. M., Gruppen, H., & Van Boekel, M. A. (2005). Gelation behavior of protein isolates extracted from 5 cultivars of Pisum sativum L. Journal of Food Science, 70(2), C132-C137. https://doi.org/10.1111/j.1365-2621.2005.tb07073.x.
Oehmigen, K., Hähnel, M., Brandenburg, R., Wilke, C., Weltmann, K. D., & Von Woedtke, T. (2010). The role of acidification for antimicrobial activity of atmospheric pressure plasma in liquids. Plasma Processes and Polymers, 7(3-4), 250-257.
Pankaj, S., Misra, N., & Cullen, P. (2013). Kinetics of tomato peroxidase inactivation by atmospheric pressure cold plasma based on dielectric barrier discharge. Innovative Food Science & Emerging Technologies, 19, 153-157.
Picout, D. R., & Ross-Murphy, S. B. (2003). Rheology of biopolymer solutions and gels. The Scientific World Journal, 3, 105-121. https://doi.org/10.1100/tsw.2003.15.
Pownall, T. L., Udenigwe, C. C., & Aluko, R. E. (2010). Amino acid composition and antioxidant properties of pea seed (Pisum sativum L.) enzymatic protein hydrolysate fractions. Journal of Agricultural and Food Chemistry, 58(8), 4712-4718. https://doi.org/10.1021/jf904456r.
Schmidt, V., Giacomelli, C., & Soldi, V. (2005). Thermal stability of films formed by soy protein isolate-sodium dodecyl sulfate. Polymer Degradation and Stability, 87(1), 25-31. https://doi.org/10.1016/j.polymdegradstab.2004.07.003.
Services, H. a. H. (2017). Food safety when cooking. In. [symbol]
Shand, P. J., Ya, H., Pietrasik, Z., & Wanasundara, P. K. J. P. D. (2007). Physicochemical and textural properties of heat-induced pea protein isolate gels. Food Chemistry, 102(4), 1119-1130. https://doi.org/10.1016/j.foodchem.2006.06.060.
Shand, P. J., Ya, H., Pietrasik, Z., & Wanasundara, P. K. J. P. D. (2008). Transglutaminase treatment of pea proteins: Effect on physicochemical and rheological properties of heat-induced protein gels. Food Chemistry, 107(2), 692-699. https://doi.org/10.1016/j.foodchem.2007.08.095.
Sullivan, G. A., & Sebranek, J. G (2012). Nitrosylation of myoglobin and nitrosation of cysteine by nitrite in a model system simulating meat curing. Journal of Agricultural and Food Chemistry, 60(7), 1748-1754. https://doi.org/10.1021/jf204717v.
Sun, X. D., & Arntfield, S. D. (2010). Gelation properties of salt-extracted pea protein induced by heat treatment. Food Research International, 43(2), 509-515. https://doi.org/10.1016/j.foodres.2009.09.039.
Sun, X. D., & Arntfield, S. D. (2011a). Dynamic oscillatory rheological measurement and thermal properties of pea protein extracted by salt method: Effect of pH and NaCl. Journal of Food Engineering, 105(3), 577-582. (a) https://doi.org/10.1016/j.jfoodeng.2011.03.008.
Sun, X. D., & Arntfield, S. D. (2011b). Gelation properties of salt-extracted pea protein isolate induced by heat treatment: Effect of heating and cooling rate. Food Chemistry, 124(3), 1011-1016. (b) https://doi.org/10.1016/j.foodchem.2010.07.063.
Sun, X. D., & Arntfield, S. D. (2012). Molecular forces involved in heat-induced pea protein gelation: effects of various reagents on the rheological properties of salt-extracted pea protein gels. Food Hydrocolloids, 28(2), 325-332. https://doi.org/10.1016/j.foodhyd.2011.12.014.
Taherian, A. R., Mondor, M., Labranche, J., Drolet, H., Ippersiel, D., & Lamarche, F. (2011). Comparative study of functional properties of commercial and membrane processed yellow pea protein isolates. Food Research International, 44(8), 2505-2514. https://doi.org/10.1016/j.foodres.2011.01.030.
Tayeh, N., Aubert, G, Pilet-Nayel, M.-L., Lejeune-Hénaut, I., Warkentin, T. D., & Burstin, J. (2015). Genomic tools in pea breeding programs: status and perspectives. Frontiers in Plant Science, 6, 1037. https://doi.org/10.3389/fpls.2015.01037.
Tsumura, K., Saito, T., Tsuge, K., Ashida, H., Kugimiya, W., & Inouye, K. (2005). Functional properties of soy protein hydrolysates obtained by selective proteolysis. LWT—Food Science and Technology, 38(3), 255-261. https://doi.org/10.1016/j.lwt.2004.06.007.
Tunick, M. H. (2011). Small-strain dynamic rheology of food protein networks. Journal of Agricultural and Food Chemistry, 59(5), 1481-1486. https://doi.org/10.1021/jf1016237.
Walia, N., & Chen, L. (2020). Pea protein based vitamin D nanoemulsions: Fabrication, stability and in vitro study using Caco-2 cells. Food Chemistry, 305, 125475. https://doi.org/10.1016/j.foodchem.2019.125475.
Wang, K., & Arntfield, S. D. (2014). Binding of carbonyl flavours to canola, pea and wheat proteins using GC/MS approach. Food Chemistry, 157, 364-372. https://doi.org/10.1016/j.foodchem.2014.02.042.
Wang, K.-Q., Luo, S.-Z., Zhong, X.-Y., Cai, J., Jiang, S.-T., & Zheng, Z. (2017). Changes in chemical interactions and protein conformation during heat-induced wheat gluten gel formation. Food Chemistry, 214, 393-399. https://doi.org/10.1016/j.foodchem.2016.07.037.
Wang, Q., Jin, Y., & Xiong, Y. L. (2018). Heating-aided pH shifting modifies hemp seed protein structure, cross-linking, and emulsifying properties. Journal of Agricultural and Food Chemistry, 66(41), 10827-10834.
Wang, R., Tian, Z., & Chen, L. (2011). Nano-encapsulations liberated from barley protein microparticles for oral delivery of bioactive compounds. International Journal of Pharmaceutics, 406(1-2), 153-162.
Wang, Y., Yang, F., Wu, M., Li, J., Bai, Y., Xu, W., & Qiu, S. (2020). Synergistic effect of pH shifting and mild heating in improving heat induced gel properties of peanut protein isolate. LWT—Food Science and Technology, 131, 109812.
Wu, M., Xiong, Y. L., Chen, J., Tang, X., & Zhou, G (2009). Rheological and microstructural properties of porcine myofibrillar protein-lipid emulsion composite gels. Journal of Food Science, 74(4), E207-E217. https://doi.org/10.1111/j.1750-3841.2009.01140.x.
Xiong, T., Xiong, W., Ge, M., Xia, J., Li, B., & Chen, Y. (2018). Effect of high intensity ultrasound on structure and foaming properties of pea protein isolate. Food Research International, 109, 260-267.
Yan, X., Yang, J., Chen, F., Zhu, L., Tang, Z., Qin, G, Chen, Q., & Chen, G (2018). Mechanical properties of gelatin/polyacrylamide/graphene oxide nanocomposite double-network hydrogels. Composites Science and Technology, 163, 81-88. https://doi.org/10.1016/j.compscitech.2018.05.011.
Yang, C., Wang, Y., & Chen, L. (2017). Fabrication, characterization and controlled release properties of oat protein gels with percolating structure induced by cold gelation. Food Hydrocolloids, 62, 21-34.
Yang, C., Wang, Y., Vasanthan, T., & Chen, L. (2014). Impacts of pH and heating temperature on formation mechanisms and properties of thermally induced canola protein gels. Food Hydrocolloids, 40, 225-236. https://doi.org/10.1016/j.foodhyd.2014.03.011.
Yang, J., Liu, G., Zeng, H., & Chen, L. (2018). Effects of high pressure homogenization on faba bean protein aggregation in relation to solubility and interfacial properties. Food Hydrocolloids, 83, 275-286.
Yildiz, G, Andrade, J., Engeseth, N. E., & Feng, H. (2017). Functionalizing soy protein nano-aggregates with pH-shifting and mano-thermo-sonication. Journal of Colloid and Interface Science, 505, 836-846.
Yu, Y., Guan, Y., Liu, J., Hedi, W., Yu, Y., & Zhang, T. (2021). Molecular structural modification of egg white protein by pH-shifting for improving emulsifying capacity and stability. Food Hydrocolloids, 121, 107071.
Zhang, D., Li, H., Emara, A., Hu, Y., Wang, Z., Wang, M., & He, Z. (2020). Effect of in vitro oxidation on the water retention mechanism of myofibrillar proteins gel from pork muscles. Food Chemistry, 315, 126226. https://doi.org/10.1016/j.foodchem.2020.126226.
Zhang, S., Huang, W., Feizollahi, E., Roopesh, M., & Chen, L. (2020). Improvement of pea protein gelation at reduced temperature by atmospheric cold plasma and the gelling mechanism study. Innovative Food Science & Emerging Technologies, 102567.
Zhang, T., Jiang, B., & Wang, Z. (2007). Gelation properties of chickpea protein isolates. Food Hydrocolloids, 21(2), 280-286.
Zhang, W., Liu, C., Zhao, J., Ma, T., He, Z., Huang, M., & Wang, Y. (2021). Modification of structure and functionalities of ginkgo seed proteins by pH-shifting treatment. Food Chemistry, 358, 129862.
Zhao, H., Yu, B., Hemar, Y., Chen, J., & Cui, B. (2020). Improvement of calcium sulfate-induced gelation of soy protein via incorporation of soy oil before and after thermal denaturation. LWT—Food Science and Technology, 117, 108690.
Zhou, G, Liu, J., Wang, G, Wang, L., Zhang, A., Wang, Y., & Wang, X. (2019). Effect of Ultrasonic Treatment on Freeze-thaw Stability of Soy Protein Isolate Gel. Journal of Oleo Science, ess19167. https://doi.org/10.5650/jos.ess19167.
Zhou, R., Zhou, R., Prasad, K., Fang, Z., Speight, R., Bazaka, K., & Ostrikov, K. K. (2018). Cold atmospheric plasma activated water as a prospective disinfectant: the crucial role of peroxynitrite. Green Chemistry, 20(23), 5276-5284.

1. A method of treating a pulse protein to produce a pulse protein g.el, comprising the steps of:
(a) treating the pulse protein
(i) with atmospheric cold plasma (ACP); or
(ii) by shifting to an alkaline pH greater than pH 9.0 or an acidic pH less than about pH 5.0, followed by adjusting, the pH back to neutral; or
(iii) with both ACP and pH shifting.

2. The method of claim 1 wherein the pulse protein comprises protein extracted ftom peas (Pisum sativum), chickpeas, lentils, or beans.

3. The method of claim 1. wherein the pulse protein is treated by shifting to the alkaline pH, optionally with ACP treatment, either sequentially in either order or simultaneously.

4. The method of claim 1, wherein the alkaline pH is between about pH 10.0 to about: 12.0.

5. The method of claim 3, wherein the protein is held at the alkaline pH for longer than about 1 hour.

6. The method of claim 5 wherein the protein is held at the alkaline pH for about 48 hours.

7. The method of claim 1, wherein the length of ACP treatment is greater than about 2 minutes and less than about 20 minutes.,

8. The method of claim 1. wherein the protein is treated by both ACP and pH shift to about pH 12.0.

9. The method of claim 1. wherein the protein is treated by both ACP and pH shift with a total treatment time of between about 2 to about 20 minutes.

10. The method of claim 1, comprising the thither step of heating the protein to induce gelation.

11. The method of claim 10 wherein a. minimum gel compressive strength of 1.0 kPa. 2.0 kPa, or 3.0 kPa is achieved without incorporation of an additional cross-linking agent or non-pulse protein.

12. The method of claim 10 wherein the gel is formed at a gelation temperature below about 95° C., about 85° C. or about 80° C.

13. A protein gel comprising a pulse protein treated with a method of claim 1.

14. A food additive comprising pulse protein which has been treated in accordance with claim 1.

15. The additive of claim 14 which is a powder or a liquid suspension.

16. The additive of claim 14 wherein the pulse protein comprises protein extracted from peas (Pisum sativum), chickpeas, lentils, or beans.
